(19) United States
(12) Patent Application Publication
(10) Pub. No.: US 2023/0106836 A1
(43) Pub. Date: Apr. 6, 2023

(54) METHOD OF USING BIOSURFACTANT-PRODUCING BACTERIA AGAINST FUNGAL AND BACTERIAL PATHOGENS

(71) Applicant: THE ROYAL INSTITUTION FOR THE ADVANCEMENT OF LEARNING/MCGILL UNIVERSITY, Montreal (CA)

(72) Inventor: Suha JABAJI, Notre Dame de l'ile Perrot (CA)

(21) Appl. No.: 17/904,666

(22) PCT Filed: Feb. 19, 2021
(86) PCT No.: PCT/CA2021/050194

§ 371 Date: Aug. 19, 2022

Related U.S. Application Data
(60) Provisional application No. 62/979,570, filed on Feb. 21, 2020.

(51) Int. Cl. A01N 63/22; C12N 1/20; A01N 63/28; A01N 63/27; A01N 63/20; A01N 63/50; A01P 1/00; A23B 7/155; A23B 7/16

(52) U.S. Cl. CPC A01N 63/22; C12N 1/205; A01N 63/28; A01N 63/27; A01N 63/20; A01N 63/50; A01P 1/00; A23B 7/155; A23B 7/16; C12R 2001/07

(57) ABSTRACT

The disclosure provides a method of using a bacterium that is a Bacillus, Streptomyces, Microbacterium, Micrococcus, Rhodococcus, Pseudomonas, Arthrobacter or Staphylococcus and/or a biosurfactant-containing extract isolated from said bacterium, as an antimicrobial agent against a foodborne or a plant bacterial or fungal pathogen, wherein the bacterium optionally comprises at least 3 of bacilysin, difficidin, macrolactin h, bacillaene, bacillomycin d, fengycin, surfactin and bacillibactin. The disclosure also provides a plant or plant part bacterized or coated with a Bacillus or a biosurfactant-containing extract isolated from the Bacillus and a method of protecting a plant or plant part against a bacterial or fungal pathogen comprising bacterizing or coating the plant or plant part with a Bacillus or a biosurfactant-containing extract isolated from the Bacillus, wherein the Bacillus produces or the extract contains at least 3 of bacilysin, difficidin, macrolactin h, bacillaene, bacillomycin d, fengycin, surfactin and bacillibactin.

### CROSS REFERENCE TO RELATED APPLICATIONS

This application is a PCT application filed on Feb. 22, 2021 and published in English under PCT Article 21(2), which itself claims benefit of U.S. provisional application Ser. No. 62/979,570, filed on Feb. 21, 2021. All documents above are incorporated herein in their entirety by reference.

### STATEMENT REGARDING FEDERALLY SPONSORED RESEARCH OR DEVELOPMENT

N.A.

### FIELD OF THE DISCLOSURE

The present disclosure relates to method of using biosurfactant-producing bacteria against bacterial and fungal pathogens. More specifically, the present disclosure is concerned with biosurfactant-producing actinobacteria, proteobacteria and firmicutes and their use against foodborne and plant bacterial and fungal pathogens.

### REFERENCE TO SEQUENCE LISTING

Pursuant to 37 C.F.R. 1.821(c), a sequence listing is submitted herewith as an ASCII compliant text file named Sequence Listing 11168-00483_ST25, that was created on Feb. 18, 2021 and having a size of 1009 kilobytes. The content of the aforementioned file named Sequence Listing 11168-00483_ST25 is hereby incorporated by reference in its entirety.

### BACKGROUND OF THE DISCLOSURE

A number of studies have demonstrated the ability of human pathogenic bacteria to colonize not only the surface of the host plant, but also the interior; thereby raising concerns of contracting foodborne illness from vegetables (Fletcher et al., 2013). Recently reported outbreaks of E. coli O157:H7 on fresh lettuce and spinach and Salmonellosis from hot peppers underscore these concerns (Fletcher et al., 2013). High numbers of Salmonella enterica Newport SL1 have also been noted to adhere to alfalfa sprouts, which was directly related to the ability to produce aggregate curli fimbriae (Barak et al., 2002).

Some of the leading causes of foodborne illness are Salmonella and E. coli O157:H7 (Lynch et al., 2009). The Salmonellosis and E. coli O157:H7 outbreaks associated with fresh produce have been attributed most frequently to consumption of tomatoes, cantaloupes and leafy greens (Murray et al., 2017). Edible plants that become contaminated during agricultural practices can transmit the pathogen to consumers leading possibly to Salmonellosis outbreaks (Snyder et al., 2019). Mounting evidence indicates that foodborne pathogens not only contaminate plant surfaces but are also able to internalize into the tissues of fresh produce during the growing and the distribution processes (Deering et al., 2012). Thus, reducing Salmonella enterica association with plants during crop production could reduce risks of fresh produce-borne Salmonellosis.

In addition to being a vector of foodborne disease, fresh vegetables are of economic concern with respect to pathogens that directly result in destruction of the plant and its resultant yield. One of the most devastating diseases of lettuce is bacterial leaf spot (BLS) disease, caused by Xanthomonas campestris. Water soaked lesions appear on the margins of the leaves, reducing the quality and affecting the market value of lettuce (Nicolas et al., 2018). In Quebec, severe outbreaks of BLS can lead to devastating economic losses (i.e., 100% of the lettuce in the field is ruined).

There is a need for additional agents against human/plant pathogens.

The present description refers to a number of documents, the content of which is herein incorporated by reference in their entirety.

### SUMMARY OF THE DISCLOSURE

The present disclosure identified newly characterized microbes with prophylactic abilities against foodborne and plant pathogens. Foodborne bacteria, such as Salmonella strains, are known to exist in biofilms, which consist of the bacteria embedded in an extracellular matrix that enhances adherence and persistence to/on abiotic and biotic surfaces, including fresh produce and other surfaces along the food supply chain (Stepanović et al., 2004). The biofilm matrix is self-produced by the embedded bacteria and consists predominantly of a mixture of protein, carbohydrate and nucleic acid material. For example, many Salmonella and E. coli strains can produce the adhesin curli fimbriae, and the exopolysaccharide, cellulose, that are involved in surface adhesion, cell aggregation and persistence of these bacteria in various environments (Zogaj et al., 2001). Loss or disruption of any of these components leads to distinct colony morphotypes and decreased persistence/survival outside human hosts or on plants, such as fruits and vegetables (Milanov et al., 2015).

The present disclosure characterizes bacterial strains isolated from crude-oil (petroleum) samples and demonstrated their biosurfactant-producing potential using multiple screening methods.

Biosurfactants are surface-active compounds with emulsifying activities. These compounds are described as amphiphilic, containing both hydrophilic and hydrophobic ends, that allows them to interact at the interface between aqueous and non-aqueous systems (Marchant et al., 2012). There are six classes of biosurfactants: glycolipids, lipopeptides or lipoproteins, neutral lipids, phospholipids, substituted fatty acids and lipopolysaccharides. The class of biosurfactant that the genus Bacillus produces is lipopeptides (Zhao et al., 2017). Microbial biosurfactants have advantages (e.g., low toxicity to the environment, eco-friendly, biodegradability and acceptability) over their synthetic counterparts that makes them amenable for application in the fields of agriculture, the food industry and therapeutics (Mani et al., 2016; Nitschke et al., 2007; Sachdev et al., 2013).

More particularly, 123 bacterial strains were isolated from three oil batteries in the region of Chauvin, Alberta, and characterized by 16S rRNA gene sequencing. Based on their nucleotide sequences, the strains are associated with 3 phyla (Actinobacteria, Proteobacteria and Firmicutes), as well as 17 other discrete genera that shared high homology with known sequences, with the majority of these strains identified to the species level. The most prevalent strains associated with the three oil wells belonged to the Bacillus genus. Thirty-four of the 123 strains were identified as biosurfactant-producers, among which a specific Bacillus methylotrophicus strain OB9 exhibited the highest biosurfactant activity based on multiple screening methods and a comparative analysis with the commercially available biosurfactant, Tween™ 20.

The present disclosure shows the antimicrobial activity of B. velezensis (previously called methylotrophicus) OB9(or partially purified biosurfactant fractions thereof) in agar-diffusion assays against a panel of control Salmonella enterica Typhimurium strains displaying various biofilm expression morphotypes (UMR1, MAE14, MAE299 and MAE775); thereby indicating that the antagonistic activities of B. methylotrophicus OB9 are not circumvented by the biofilm barrier. Furthermore, the antagonistic capability of B. methylotrophicus OB9 was also noted against 17 other Salmonella serovars, including top clinical serovars Typhimurium Heidelberg, Newport, Infantis, Thompson and Braenderup, as well as environmental isolates. The activity of B. methylotrophicus OB9 also proved effective on a broader scale, where it affected 4 strains of E. coli, with the highest activity against E. coli E10-6, and a virulent strain of X. campestris, the causal agent of bacterial leaf spot on lettuce. B. methylotrophicus OB9 was antagonistic against the growth of fungal varieties as well, especially R. solani. The present disclosure demonstrates the inhibition spectrum of B. methylotrophicus OB9 against both human and plant pathogens; thereby making this bacterium attractive for agricultural and food safety applications in a climate where microbial-biofilm persistence is an increasing problem.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

In the present description, a number of terms are extensively utilized. In order to provide a clear and consistent understanding of the specification and claims, including the scope to be given such terms, the following definitions are provided.

The use of the word “a” or “an” when used in conjunction with the term “comprising” in the claims and/or the specification may mean “one” but it is also consistent with the meaning of “one or more”, “at least one”, and “one or more than one”. Throughout this specification, unless the context requires otherwise, the words “comprise,” “comprises” and “comprising” will be understood to imply the inclusion of a stated step or element or group of steps or elements but not the exclusion of any other step or element or group of steps or elements.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the term “consists of” or “consisting of” means including only the elements, steps, or ingredients specifically recited in the particular claimed embodiment or claim.

Terms and symbols of genetics, molecular biology, biochemistry and nucleic acid used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

### Bacterial Strains

The present disclosure encompasses the use of biosurfactant-producing bacterial strains as disclosed herein. In particular, it encompasses the use of bacterial strains that produce at least one of (at least 2, 3, 4, 5, 6, 7 or all 8 of) bacilysin, difficidin, macrolactin H, bacillaene, bacillomycin D, fengycin, surfactin and bacillibactin. In specific embodiments, it encompasses the use of bacterial strains isolated from oil (i.e., from crude oil/unrefined petroleum or oil-dwelling bacteria), and more particularly from the aqueous phase of a water extract of such oil. In specific embodiments, it encompasses bacterial strains (e.g., Bacillus such as Bacillus velezensis) encoding at least one of (at least 5, 10, 15, 20, 25, 30, 35, 40, 45, 50, 55, 60, 65, 70, 75, 80, 85, 90, 95, 100, 105, 110, 111 or more of) the following polypeptides and enzymes: MFS transporter; 1 biosynthesis protein BacA; cupin domain-containing protein; dihydroanticapsin 7-dehydrogenase; ATP-grasp domain-containing protein; MFS transporter; pyridoxal phosphate-dependent aminotransferase; ACP S-malonyltransferase; D-fructose-6-phosphate amidotransferase; acyl carrier protein; long-chain fatty acid--CoA ligase; SDR family oxidoreductase; SDR family NAD(P)-dependent oxidoreductase; type I polyketide synthase; KR domain-containing protein; SDR family NAD(P)-dependent oxidoreductase; SDR family NAD(P)-dependent oxidoreductase; SDR family NAD(P)-dependent oxidoreductase; SDR family NAD(P)-dependent oxidoreductase; SDR family NAD(P)-dependent oxidoreductase; zinc-binding dehydrogenase; cytochrome P450; hydroxymethylglutaryl-CoA synthase family protein; polyketide biosynthesis enoyl-CoA isomerase; ACP S-malonyltransferase; SDR family NAD(P)-dependent oxidoreductase; SDR family NAD(P)-dependent oxidoreductase; SDR family NAD(P)-dependent oxidoreductase; SDR family NAD(P)-dependent oxidoreductase; SDR family NAD(P)-dependent oxidoreductase; SDR family NAD(P)-dependent oxidoreductase; alpha/beta fold hydrolase; serine hydrolase; pyruvate dehydrogenase (acetyl-transferring) E1 component subunit alpha; MBL fold metallo-hydrolase; ACP S-malonyltransferase; acyltransferase domain-containing protein; ACP S-malonyltransferase; acyl carrier protein; hydroxymethylglutaryl-CoA synthase family protein; enoyl-CoA hydratase/isomerase; enoyl-CoA hydratase; non-ribosomal peptide synthetase; SDR family NAD(P)-dependent oxidoreductase; SDR family NAD(P)-dependent oxidoreductase; non-ribosomal peptide synthetase; methyltransferase; cytochrome P450; GntP family permease; CoA transferase subunit A; CoA transferase subunit B; 3-hydroxybutyrate dehydrogenase; ACP S-malonyltransferase; non-ribosomal peptide synthetase; non-ribosomal peptide synthetase; non-ribosomal peptide synthetase; carbohydrate-binding protein; glucuronoxylanase; D-alanyl-D-alanine carboxypeptidase/D-alanyl-D-alanine-endopeptidase; non-ribosomal peptide synthetase; amino acid adenylation domain-containing protein; amino acid adenylation domain-containing protein; non-ribosomal peptide synthetase; AMP-binding protein; amino acid adenylation domain-containing protein; non-ribosomal peptide synthase; DUF1360 domain-containing protein; family 10 glycosylhydrolase; acyl-CoA dehydrogenase; AMP-binding protein; acetyl-CoA carboxylase biotin carboxylase subunit; acetyl-CoA carboxylase biotin carboxyl carrier protein subunit; hydroxymethylglutaryl-CoA lyase; enoyl-CoA hydratase; acyl-CoA carboxylase subunit beta; GTP-binding protein; glutathione-dependent formaldehyde dehydrogenase; RDD family protein; YckD family protein; family 1 glycosylhydrolase; inhibitor of the DNA degrading activity of NucA (competence); DNA-entry nuclease; 6-phospho-3-hexuloisomerase; 3-hexulose-6-phosphate synthase; winged helix-turn-helix transcriptional regulator; 7 non-ribosomal peptide synthetase SrfAA; 7 non-ribosomal peptide synthetase SrfAB; AMP-binding protein; 7 non-ribosomal peptide synthetase SrfAC; 7 biosynthesis thioesterase SrfAD; aminotransferase class I/II-fold pyridoxal phosphate-dependent enzyme; YcxB family protein; DMT family transporter; PLP-dependent aminotransferase family protein; 4′-phosphopantetheinyl transferase superfamily protein; YitT family protein; cystine ABC transporter ATP-binding protein TcyC; amino acid ABC transporter permease; leucyl aminopeptidase; TRAP transporter large permease subunit; biotin transporter BioY; sulfite oxidase-like oxidoreductase; alpha/beta hydrolase; 2,3-dihydro-2,3-dihydroxybenzoate dehydrogenase; isochorismate synthase DhbC; (2,3-dihydroxybenzoyl)adenylate synthase; isochorismatase; non-ribosomal peptide synthetase; MbtH family protein; YukJ family protein; alanine dehydrogenase. In more specific embodiments, it encompasses bacterial strains that encode the gene products that participate in the synthesis of at least one of (at least 2, 3, 4, 5, 6,7 or all 8 of) bacilysin, difficidin, macrolactin H, bacillaene, bacillomycin D, fengycin, surfactin and bacillibactin. Without being so limited, such gene products are listed in Table I below. In more specific embodiments, it encompasses bacterial strains that encode at least one protein having an amino acid sequence as set forth in any one of SEQ ID NOs: 1-111. Without being so limited, the biosurfactant-producing bacterial strain is a Bacillus, Streptomyces, Microbacterium, Micrococcus, Rhodococcus, Pseudomonas, Arthrobacteror Staphylococcus (e.g., that is isolated from crude oil/oil dwelling bacterial strain). Without being so limited, the biosurfactant-producing bacterial strain is of one of the species listed in Table IV below. Without being so limited, the biosurfactant-producing bacterial strain is a Bacillus velezensis or a Bacillus amyloliquefaciens. Without being so limited, the biosurfactant-producing bacterial strain is a Bacillus velezensis (e.g., that is isolated from crude oil/oil dwelling bacterial strain).

As used herein, the term “biosurfactant-containing extract” refers to an extract of a bacterium (or bacteria) as disclosed herein comprising at least one biosurfactant. The extract is a whole extract or a fraction thereof which is obtained through any process known in the art for extracting biosurfactant from a bacterium or bacteria. Another commonly used process comprises using chloroform: methanol v:v (2:1) as the extraction solvent, followed by ethyl acetate and methanol. Without being so limited it includes supernatant of bacterial cell medium and acid fractions thereof such as but not limited to acid precipitate fraction.

### Forms of the Bacteria of the Present Invention

Although the bacteria (e.g., Bacillus such as Bacillus velezensis) of the present invention is an effective antibiotic against foodborne, fecal and environment human and plant pathogens when used alone (i.e. as a biologically pure strain), it may nevertheless also be used in combination with other bacteria (e.g., biosurfactant-producing or other). The present invention encompasses the use of the Bacillus methylotrophicus of the present invention as sole antibiotic bacteria or in combination with one or more other useful bacteria.

As used herein, the terminology “biologically pure” strain is intended to mean a strain separated from materials with which it is normally associated in nature. Note that a strain associated with compounds or materials that it is not normally found with in nature, is still defined as “biologically pure”. A monoculture of a particular strain is, of course, “biologically pure.”

For the methods and uses of the present invention, it is not necessary that the whole broth culture of the strains of the invention be used. Indeed, the present invention encompasses the use of a whole broth culture of a strain of the present invention, endospores produced by such strain, dried biomass of the strains and lyophilized strains. As used herein therefore, the terminology application of the “bacterium” of the present invention refers to application of any form or part of the strain of the present invention or a combination thereof that possesses the desired ability to induce drought tolerance.

The bacterium (e.g., Bacillus such as Bacillus velezensis) of the present invention (e.g., OB9) can take the form of a bacterium (such as whole broth culture of a strain of the present invention, endospores produced by such strain, dried biomass of the strains and lyophilized strains), a seed of a second or subsequent (up to fourth but preferably second) generation infected with the bacterium (e.g., Bacillus such as Bacillus velezensis), or a composition comprising the bacterium (e.g., Bacillus such as Bacillus velezensis). The bacterium (e.g., Bacillus such as Bacillus velezensis) of the present invention (e.g., OB9), or a composition thereof may be applied to soil directly prior to seeding the plant or after planting the plant (as described e.g., at Example 1), sprayed (e.g., whole broth culture) on the plant, soil and/or on the seed of the plant. Said seed may be applied to soil directly.

There is also provided a combination of an inoculum of a strain according to the present invention and of one or more carriers to form a composition. Formulating the bacterium (e.g., Bacillus such as Bacillus velezensis) in a composition may increase its potential storage time and stability.

In order to achieve good dispersion, adhesion and conservation/stability of compositions within the present invention, it may be advantageous to formulate the bacterium (e.g., Bacillus such as Bacillus velezensis) (such as whole broth culture of a strain of the present invention, endospores produced by such strain, dried biomass of the strains and lyophilized strains) with components that aid dispersion, adhesion and conservation/stability of the plant on which it is applied. It could be formulated as a spray, granules or as a coating for the plant seed. These components are referred to herein individually or collectively as “carrier”. Suitable formulations for this carrier will be known to those skilled in the art (wettable powders, granules and the like, or carriers within which the inoculum can be microencapsulated in a suitable medium and the like, liquids such as aqueous flowables and aqueous suspensions, and emulsifiable concentrates).

Peat-based inoculant represents a widely form of formulation, but it is not a sustainable solution as peat is a non-renewable material (Xavier, Holloway et al. 2004). Alternative methods such as the encapsulation of microorganism with biopolymer are encompassed has alternative formulation methods (Xavier, Holloway et al. 2004, John, Tyagi et al. 2011). Encapsulation is the process of making a protective capsule around the microorganism. The matrix of microsphere protects the cells by providing pre-defined and constant microenvironment thus allowing the cells to survive and maintain metabolic activity for extended period of time. Microsphere can provide a control release of microorganism as well as serve as energy source for the microorganism from its degradation. Different natural polysaccharides and protein co-extruded with calcium alginate in order to form a gelled matric, matrix material such as starches, maltodextrin, gum Arabic, pectin, chitosan, alginate and legumes protein are also encompassed by the present invention (Khan, Korber et al. 2013, Nesterenko, Alric et al. 2013). Without being so limited, useful carriers for the present invention include propylene glycol alginate, powder or granular inert materials may include plant growth media or matrices, such as rockwool and peat-based mixes, attapulgite clays, kaolinic clay, montmorillonites, saponites, mica, perlites, vermiculite, talc, carbonates, sulfates, oxides (silicon oxides), diatomites, phytoproducts, (ground grains, pulses flour, grain bran, wood pulp, and lignin), synthetic silicates (precipitated hydrated calcium silicates and silicon dioxides, organics), polysaccharides (gums, starches, seaweed extracts, alginates, plant extracts, microbial gums), and derivatives of polysaccharides, proteins, such as gelatin, casein, and synthetic polymers, such as polyvinyl alcohols, polyvinyl pyrrolidone, polyacrylates (Date and Roughley, 1977; Dairiki and Hashimoto, 2005; Jung et al., 1982). The carrier may include components such as chitosan, vermiculite, compost, talc, milk powder, gel, etc. Other suitable formulations will be known to those skilled in the art.

Without being so limited, endospores of the present invention can be incorporated in a seed coating where the material of seed coating could be as described above, e.g., biochar, peat moss, and other biopolymer carriers e.g., activated charcoal and lignosulfonate.

As used herein, the terminology “amount effective” or “effective amount” is meant to refer to an amount sufficient to effect beneficial or desired results. An effective amount can be provided in one or more administrations. In terms inducing antimicrobial activity against bacterial pathogen in plant, an “effective amount” of the microorganism of the present invention is an amount sufficient to effect antimicrobial activity in a plant as compared to that exhibited by plant in the absence of the microorganism. In a specific embodiment, it refers to an amount of about 1×10⁸ CFU or more/plant, plant part, or area around a plant or plant part.

### Plants

Plants benefiting from the bacterium (e.g., Bacillus such as Bacillus velezensis) of the present invention include any horticultural or agricultural plant. Without being so limited, it refers to any plant that can be commercialized including comestible plants.

In a specific embodiment, the plant is of the clade asterid. In a more specific embodiment, the asterid plant is of the solanales order. In another more specific embodiment, the solanales plant is of the solanaceae family (such as capsicum i.e. pepper). In another more specific embodiment, the solanales plant is of the Solanum genus (such as tomato, potato, eggplant, illustrated herein with tomato).

In another more specific embodiment, the asterid plant is of the asterales order. In another more specific embodiment, the asterales plant is of the Asteraceae family. In another more specific embodiment, the Asteraceae plant is of the cichorieae tribe. In another more specific embodiment, the cichorieae plant is of the Lactuca genus (illustrated herein with lettuce)

As used herein, the term “plant part” refers to any plant part removed from a growing or grown plant (in soil or other growing media, such as hydroponic, aquaponic, etc.). Without being so limited, it refers to any plant part that can be commercialized including comestible plant parts such as fruits and vegetables, shoots, leaves, stems, flowers, seeds and roots.

### Application

As indicated above, the methods of the present invention comprise applying the bacterium (e.g., Bacillus such as Bacillus velezensis) or a composition thereof (i) to the plant or to a part of the plant; and/or (ii) to an area around the plant or plant part. The treated seeds can be planted thereafter and grown into a plant that exhibits the ability to resist to bacterial infection. As used herein the terms “area around the plant or plant part” refers to the soil or plant pot prior to planting the plant seedling or seed, or during or after having planted the plant seedling or seed.

As used herein the term “coating” in the context of coating a plant or plant part with a bacterium, bacteria or biosurfactant-containing extract isolated from the bacterium or bacteria refers to applying on the surface of the plant or plant part or plant growth media.

As used herein the term “bacterizing” refers to any techniques used to enable the colonization of a plant by the bacterium. For this the bacterium (in a culture media or otherwise) can be applied on a seed or a seedling. Without being so limited, it includes applying the bacterium on the growth media (e.g., soil, water) close to the roots of the plant's seedling to enable the colonization of the plant by the bacterium.

TABLE I
Gene description and gene products of Bacillus velezensis OB9

| | | | NCBI PGAP | CONTIG | | | |
|---|---|---|---|---|---|---|---|
| | | MIBIG BGC GENE | ANNOTATED | ID^{B} | FROM | TO | SEQ |
| ^{a} | LOCUS TAG | NAME/DESCRIPTION | PRODUCT FUNCTION | # | (BP) | (BP) | NO: |
| 1 | AC810_RS02580 | YwfA | MFS transporter | 1 | 517903 | 525201 | 1. |
| 1 | AC810_RS02575 | BacA | 1 biosynthesis protein | 1 | 523187 | 523801 | 2. |
| | | | BacA | | | | |
| 1 | AC810_RS02570 | BacB | cupin domain-containing | 1 | 522487 | 523197 | 3. |
| | | | protein | | | | |
| 1 | AC810_RS02565 | BacC | dihydroanticapsin 7- | 1 | 521729 | 522490 | 4. |
| | | | dehydrogenase | | | | |
| 1 | AC810_RS02560 | BacD | ATP-grasp domain- | 1 | 520293 | 521711 | 5. |
| | | | containing protein | | | | |
| 1 | AC810_RS02555 | BacE | MFS transporter | 1 | 519115 | 520296 | 6. |
| 1 | AC810_RS02550 | YwfG | pyridoxal phosphate- | 1 | 517903 | 519102 | 7. |
| | | | dependent | | | | |
| | | | aminotransferase | | | | |
| 2 | AC810_RS06775 | malonyl CoA-acyl | ACP S- | 2 | 562177 | 564435 | 8. |
| | | carrier protein | malonyltransferase | | | | |
| | | transacylase | | | | | |
| 2 | AC810_RS06780 | putative long-chain | D-fructose-6-phosphate | 2 | 564476 | 565456 | 9. |
| | | fatty_acid CoA ligase | amidotransferase | | | | |
| 2 | AC810_RS06785 | probable acyl carrier | acyl carrier protein | 2 | 565481 | 565753 | 10. |
| | | protein | | | | | |
| 2 | AC810_RS06790 | acyl CoA synthetase | long-chain fatty acid- | 2 | 565750 | 567114 | 11. |
| | | (AMP forming)/AMP | CoA ligase | | | | |
| | | acid ligase II | | | | | |
| 2 | AC810_RS06795 | 3-oxoacyl-(acyl carrier | SDR family | 2 | 567129 | 567866 | 12. |
| | | protein) reductase | oxidoreductase | | | | |
| 2 | AC810_RS06800 | polyketide synthase | SDR family NAD(P)- | 2 | 567906 | 577193 | 13. |
| | | type I | dependent | | | | |
| | | | oxidoreductase | | | | |
| 2 | AC810_RS18325 | polyketide synthase | type I polyketide | 15 | 2 | 982 | 14. |
| | | type I | synthase | | | | |
| 2 | AC810_RS18330 | polyketide synthase | KR domain-containing | 15 | 1001 | 7297 | 15. |
| | | type I | protein | | | | |
| 2 | AC810_RS18335 | polyketide synthase | SDR family NAD(P)- | 15 | 7337 | 13063 | 16. |
| | | type I | dependent | | | | |
| | | | oxidoreductase | | | | |
| 2 | AC810_RS18340 | polyketide synthase | SDR family NAD(P)- | 15 | 13115 | 24961 | 17. |
| | | type I | dependent | | | | |
| | | | oxidoreductase | | | | |
| 2 | AC810_RS10990 | polyketide synthase | SDR family NAD(P)- | 5 | 2 | 6358 | 18. |
| | | type I | dependent | | | | |
| | | | oxidoreductase | | | | |
| 2 | AC810_RS10995 | polyketide synthase | SDR family NAD(P)- | 5 | 6363 | 14081 | 19. |
| | | type I | dependent | | | | |
| | | | oxidoreductase | | | | |
| 2 | AC810_RS11000 | polyketide synthase | SDR family NAD(P)- | 5 | 14104 | 20256 | 20. |
| | | type I | dependent | | | | |
| | | | oxidoreductase | | | | |
| 2 | AC810_RS11005 | polyketide synthase | zinc-binding | 5 | 20253 | 26468 | 21. |
| | | type I | dehydrogenase | | | | |
| 2 | AC810_RS11010 | putative cytochrome | cytochrome P450 | 5 | 26550 | 27704 | 22. |
| | | P450 monooxygenase | | | | | |
| 2 | AC810_RS11015 | 3-hydroxy-3- | hydroxymethylglutaryl- | 5 | 27762 | 29009 | 23. |
| | | methylglutaryl | CoA synthase family | | | | |
| | | coenzyme A synthase- | protein | | | | |
| | | like | | | | | |
| 2 | AC810_RS11020 | Enoyl-CoA hydratase | polyketide biosynthesis | 5 | 29069 | 29815 | 24. |
| | | | enoyl-CoA isomerase | | | | |
| 3 | AC810_RS08985 | malonyl CoA [acyl- | ACP S- | 3 | 411925 | 414231 | 25. |
| | | carrier-protein] | malonyltransferase | | | | |
| | | transacylase | | | | | |
| 3 | AC810_RS08990 | polyketide synthase | SDR family NAD(P)- | 3 | 414253 | 426507 | 26. |
| | | type I | dependent | | | | |
| | | | oxidoreductase | | | | |
| 3 | AC810_RS08995 | polyketide synthase | SDR family NAD(P)- | 3 | 426507 | 431279 | 27. |
| | | type I | dependent | | | | |
| | | | oxidoreductase | | | | |
| 3 | AC810_RS09000 | polyketide synthase | SDR family NAD(P)- | 3 | 431327 | 440035 | 28. |
| | | type I | dependent | | | | |
| | | | oxidoreductase | | | | |
| 3 | AC810_RS09005 | polyketide synthase | SDR family NAD(P)- | 3 | 440028 | 447032 | 29. |
| | | type I | dependent | | | | |
| | | | oxidoreductase | | | | |
| 3 | AC810_RS09010 | polyketide synthase | SDR family NAD(P)- | 3 | 447056 | 452767 | 30. |
| | | type I | dependent | | | | |
| | | | oxidoreductase | | | | |
| 3 | AC810_RS09015 | polyketide synthase | SDR family NAD(P)- | 3 | 452767 | 460149 | 31. |
| | | type I | dependent | | | | |
| | | | oxidoreductase | | | | |
| 3 | AC810_RS09020 | polyketide synthase | alpha/beta fold | 3 | 460200 | 464051 | 32. |
| | | type I | hydrolase | | | | |
| 3 | AC810_RS09025 | pbp related beta- | serine hydrolase | 3 | 464084 | 465175 | 33. |
| | | lactamase precursor | | | | | |
| 3 | AC810_RS09030 | pyruvate | pyruvate dehydrogenase | 3 | 465645 | 466760 | 34. |
| | | dehydrogenase (E1 | (acetyl-transferring) E1 | | | | |
| | | alpha subunit) | component subunit | | | | |
| | | | alpha | | | | |
| 4 | AC810_RS10270 | hydroxyacylglutathione | MBL fold metallo- | 4 | 221117 | 221794 | 35. |
| | | hydrolase | hydrolase | | | | |
| 4 | AC810_RS10275 | malonyl CoA [acyl- | ACP S- | 4 | 222109 | 222978 | 36. |
| | | carrier-protein] | malonyltransferase | | | | |
| | | transacylase [AT] | | | | | |
| 4 | AC810_RS10280 | malonyl CoA [acyl- | acyltransferase domain- | 4 | 223115 | 224089 | 37. |
| | | carrier-protein] | containing protein | | | | |
| | | transacylase [AT] | | | | | |
| 4 | AC810_RS10285 | malonyl-CoA-[acyl- | ACP S- | 4 | 224091 | 226331 | 38. |
| | | carrier_protein] | malonyltransferase | | | | |
| | | transacylase | | | | | |
| | | [AT]/oxidoreductase | | | | | |
| | | [OR] | | | | | |
| 4 | AC810_RS10290 | acyl carrier protein | acyl carrier protein | 4 | 226397 | 226645 | 39. |
| 4 | AC810_RS10295 | 3-hydroxy-3- | hydroxymethylglutaryl- | 4 | 226697 | 226697 | 40. |
| | | methylglutaryl CoA | CoA synthase family | | | | |
| | | synthase | protein | | | | |
| 4 | AC810_RS10300 | polyketide | enoyl-CoA | 4 | 227956 | 228729 | 41. |
| | | biosynthesis enoyl- | hydratase/isomerase | | | | |
| | | CoA hydratase | | | | | |
| 4 | AC810_RS10305 | polyketide | enoyl-CoA hydratase | 4 | 228739 | 229488 | 42. |
| | | biosynthesis enoyl- | | | | | |
| | | CoA hydratase | | | | | |
| 4 | AC810_RS10310 | hybrid NRPS/PKS | non-ribosomal peptide | 4 | 229528 | 244482 | 43. |
| | | protein | synthetase | | | | |
| 4 | AC810_RS10315 | polyketide synthase of | SDR family NAD(P)- | 4 | 244484 | 257902 | 44. |
| | | type I | dependent | | | | |
| | | | oxidoreductase | | | | |
| 4 | AC810_RS10320 | polyketide synthase of | SDR family NAD(P)- | 4 | 257920 | 268455 | 45. |
| | | type I | dependent | | | | |
| | | | oxidoreductase | | | | |
| 4 | AC810_RS10325 | hybrid NRPS/PKS | non-ribosomal peptide | 4 | 268445 | 284746 | 46. |
| | | protein | synthetase | | | | |
| 4 | AC810_RS10330 | polyketide synthase of | methyltransferase | 4 | 284760 | 292217 | 47. |
| | | type I | | | | | |
| 4 | AC810_RS10335 | cytochrome P450 | cytochrome P450 | 4 | 292355 | 293566 | 48. |
| | | ‘oxidase’ | | | | | |
| 5 | AC810_RS10885 | YxjC | GntP family permease | 4 | 432329 | 433774 | 49. |
| 5 | AC810_RS10880 | ScoA | CoA transferase subunit | 4 | 431603 | 432304 | 50. |
| | | | A | | | | |
| 5 | AC810_RS10875 | ScoB | CoA transferase subunit | 4 | 430923 | 431585 | 51. |
| | | | B | | | | |
| 5 | AC810_RS10870 | YxjF | 3-hydroxybutyrate | 4 | 430125 | 430910 | 52. |
| | | | dehydrogenase | | | | |
| 5 | AC810_RS10865 | malonyl-CoA | ACP S- | 4 | 428363 | 429565 | 53. |
| | | transacylase | malonyltransferase | | | | |
| 5 | AC810_RS10860 | 5 synthetase A | non-ribosomal peptide | 4 | 416395 | 428343 | 54. |
| | | | synthetase | | | | |
| 5 | AC810_RS10855 | 5 synthetase B | non-ribosomal peptide | 4 | 400226 | 416350 | 55. |
| | | | synthetase | | | | |
| 5 | AC810_RS10850 | 5 synthetase C | non-ribosomal peptide | 4 | 392283 | 400142 | 56. |
| | | | synthetase | | | | |
| 5 | AC810_RS10845 | XynD | carbohydrate-binding | 4 | 390574 | 392109 | 57. |
| | | | protein | | | | |
| 5 | AC810_RS10840 | YnfF | glucuronoxylanase | 4 | 389241 | 390512 | 58. |
| 6 | AC810_RS12615 | DacC | D-alanyl-D-alanine | 5 | 316516 | 317991 | 59. |
| | | | carboxypeptidase/D- | | | | |
| | | | alanyl-D-alanine- | | | | |
| | | | endopeptidase | | | | |
| 6 | AC810_RS12620 | 6 synthetase A | non-ribosomal peptide | 5 | 318471 | 326105 | 60. |
| | | | synthetase | | | | |
| 6 | AC810_RS12625 | 6 synthetase C or D | amino acid adenylation | 5 | 326131 | 332856 | 61. |
| | | | domain-containing | | | | |
| | | | protein | | | | |
| 6 | AC810_RS18585 | 6 synthetase A | amino acid adenylation | 18 | 9921 | 14477 | 62. |
| | | | domain-containing | | | | |
| | | | protein | | | | |
| 6 | AC810_RS18580 | 6 synthetase B | non-ribosomal peptide | 18 | 2198 | 9895 | 63. |
| | | | synthetase | | | | |
| 6 | AC810_RS18575 | 6 synthetase C | AMP-binding protein | 18 | 1 | 2182 | 64. |
| 6 | AC810_RS10985 | 6 synthetase D | amino acid adenylation | 4 | 456293 | 460588 | 65. |
| | | | domain-containing | | | | |
| | | | protein | | | | |
| 6 | AC810_RS10980 | 6 synthetase E | non-ribosomal peptide | 4 | 452471 | 456274 | 66. |
| | | | synthase | | | | |
| 6 | AC810_RS10975 | YngL | DUF1360 domain- | 4 | 452017 | 452397 | 67. |
| | | | containing protein | | | | |
| 6 | AC810_RS10970 | YngK | family 10 | 4 | 450357 | 451895 | 68. |
| | | | glycosylhydrolase | | | | |
| 6 | AC810_RS10965 | YngJ | acyl-CoA | 4 | 449074 | 450216 | 69. |
| | | | dehydrogenase | | | | |
| 6 | AC810_RS10960 | YngI | AMP-binding protein | 4 | 447389 | 449029 | 70. |
| 6 | AC810_RS10955 | YngH | acetyl-CoA carboxylase | 4 | 446018 | 447367 | 71. |
| | | | biotin carboxylase | | | | |
| | | | subunit | | | | |
| 6 | AC810_RS10950 | hypothetical protein | acetyl-CoA carboxylase | 4 | 445809 | 446021 | 72. |
| | | | biotin carboxyl carrier | | | | |
| | | | protein subunit | | | | |
| 6 | AC810_RS10945 | YngG | hydroxymethylglutaryl- | 4 | 444894 | 445793 | 73. |
| | | | CoA lyase | | | | |
| 6 | AC810_RS10940 | YngF | enoyl-CoA hydratase | 4 | 444101 | 444880 | 74. |
| 6 | AC810_RS10935 | YngE | acyl-CoA carboxylase | 4 | 442554 | 444083 | 75. |
| | | | subunit beta | | | | |
| 7 | AC810_RS13840 | YciC protein | GTP-binding protein | 7 | 26237 | 27439 | 76. |
| 7 | AC810_RS13835 | Yx01 protein | glutathione-dependent | 7 | 25036 | 26172 | 77. |
| | | | formaldehyde | | | | |
| | | | dehydrogenase | | | | |
| 7 | AC810_RS13830 | YckC protein | RDD family protein | 7 | 24587 | 25021 | 78. |
| 7 | AC810_RS13825 | YckD protein | YckD family protein | 7 | 24192 | 24515 | 79. |
| 7 | AC810_RS13820 | YckE protein | family 1 | 7 | 22652 | 24088 | 80. |
| | | | glycosylhydrolase | | | | |
| 7 | AC810_RS13815 | Nin | inhibitor of the DNA | 7 | 22213 | 22611 | 81. |
| | | | degrading activity of | | | | |
| | | | NucA (competence) | | | | |
| 7 | AC810_RS13810 | NucA | DNA-entry nuclease | 7 | 21755 | 22192 | 82. |
| 7 | AC810_RS13805 | HxlB protein | 6-phospho-3- | 7 | 20840 | 21397 | 83. |
| | | | hexuloisomerase | | | | |
| 7 | AC810_RS13800 | HxlA protein | 3-hexulose-6-phosphate | 7 | 20208 | 20843 | 84. |
| | | | synthase | | | | |
| 7 | AC810_RS13795 | transcriptional | winged helix-turn-helix | 7 | 19614 | 19976 | 85. |
| | | regulator | transcriptional regulator | | | | |
| 7 | AC810_RS13785 | 7 synthetase A | 7 non-ribosomal peptide | 7 | 8268 | 19022 | 86. |
| | | | synthetase SrfAA | | | | |
| 7 | AC810_RS13780 | 7 synthetase B | 7 non-ribosomal peptide | 7 | 1 | 8246 | 87. |
| | | | synthetase SrfAB | | | | |
| 7 | AC810_RS13775 | 7 synthetase B | AMP-binding protein | 6 | 212875 | 215343 | 88. |
| 7 | AC810_RS13770 | 7 synthetase C | 7 non-ribosomal peptide | 6 | 209004 | 212840 | 89. |
| | | | synthetase SrfAC | | | | |
| 7 | AC810_RS13765 | 7 synthetase D | 7 biosynthesis | 6 | 208253 | 208984 | 90. |
| | | | thioesterase SrfAD | | | | |
| 7 | AC810_RS13760 | amino transferase | aminotransferase class | 6 | 206821 | 208131 | 91. |
| | | | I/II-fold pyridoxal | | | | |
| | | | phosphate-dependent | | | | |
| | | | enzyme | | | | |
| 7 | AC810_RS13755 | Not in the defined | YcxB family protein | 6 | 206247 | 206786 | 92. |
| | | duster but in this | | | | | |
| | | region of OB9 | | | | | |
| 7 | AC810_RS13750 | transporter | DMT family transporter | 6 | 205288 | 206250 | 93. |
| 7 | AC810_RS13745 | transcriptional | PLP-dependent | 6 | 203859 | 205169 | 94. |
| | | regulator containing an | aminotransferase family | | | | |
| | | aminotransferase | protein | | | | |
| | | domain | | | | | |
| 7 | AC810_RS13740 | phosphopantetheinyl | 4′-phosphopantetheinyl | 6 | 203190 | 203864 | 95. |
| | | transferase involved in | transferase superfamily | | | | |
| | | nonribosomal | protein | | | | |
| | | synthesis | | | | | |
| 7 | AC810_RS13735 | integral membrane | YitT family protein | 6 | 202444 | 203091 | 96. |
| | | protein involved in | | | | | |
| | | nonribosomal | | | | | |
| | | synthesis | | | | | |
| 7 | AC810_RS13730 | YckI protein | cystine ABC transporter | 6 | 201619 | 202362 | 97. |
| | | | ATP-binding protein | | | | |
| | | | TcyC | | | | |
| 7 | AC810_RS13725 | YckJ protein | amino acid ABC | 6 | 200902 | 201606 | 98. |
| | | | transporter permease | | | | |
| 8 | AC810_RS14880 | cytosol | leucyl aminopeptidase | 8 | 54134 | 55624 | 99. |
| | | aminopeptidase | | | | | |
| 8 | AC810_RS14885 | histidine/basic amino | TRAP transporter large | 8 | 55757 | 57085 | 100. |
| | | acid transporter | permease subunit | | | | |
| 8 | AC810_RS14890 | putative biotin | biotin transporter BioY | 8 | 57132 | 57725 | 101. |
| | | transporter | | | | | |
| 8 | AC810_RS14895 | putative molybdopterin | sulfite oxidase-like | 8 | 57825 | 58421 | 102. |
| | | containing enzyme | oxidoreductase | | | | |
| | | subunit | | | | | |
| 8 | AC810_RS14900 | 8 trilactone hydrolase | alpha/beta hydrolase | 8 | 58597 | 59466 | 103. |
| 8 | AC810_RS14905 | DhbA | 2,3-dihydro-2,3- | 8 | 59602 | 60387 | 104. |
| | | | dihydroxybenzoate | | | | |
| | | | dehydrogenase | | | | |
| 8 | AC810_RS14910 | DhbC | isochorismate synthase | 8 | 60411 | 61607 | 105. |
| | | | DhbC | | | | |
| 8 | AC810_RS14915 | DhbE | (2,3- | 8 | 61626 | 63251 | 106. |
| | | | dihydroxybenzoyl)adenyl | | | | |
| | | | -ate synthase | | | | |
| 8 | AC810_RS14920 | DhbB | isochorismatase | 8 | 63269 | 64195 | 107. |
| 8 | AC810_RS14925 | DhbF | non-ribosomal peptide | 8 | 64210 | 71337 | 108. |
| | | | synthetase | | | | |
| 8 | AC810_RS14930 | stimulator of DhbF | MbtH family protein | 8 | 71356 | 71571 | 109. |
| | | tyrosine adenylation | | | | | |
| | | activity | | | | | |
| 8 | AC810_RS14935 | conserved protein of | YukJ family protein | 8 | 71616 | 72293 | 110. |
| | | unknown function | | | | | |
| 8 | AC810_RS14940 | L-alanine | alanine dehydrogenase | 8 | 72411 | 73541 | 111. |
| | | dehydrogenase (NAD- | | | | | |
| | | dependent) | | | | | |

^{a} 1 Baciyn; 2 Difficidin; 3 Macrolactin H; 4 Bacillaene; 5 Bacillomycin D; 6 Fengycin; 7 Surfactin; 8 Bacillibactin.
^{B}NZ_LGAU0100000.

More specifically, in accordance with the present disclosure, there is provided the following items:

Item 1. Method of using a bacterium that is a Bacillus, Streptomyces, Microbacterium, Micrococcus, Rhodococcus, Pseudomonas, Arthrobacter or Staphylococcus and/or a biosurfactant-containing extract isolated from said bacterium, as an antimicrobial agent against a foodborne or a plant bacterial or fungal pathogen.

Item 2.The method of item 1, wherein the bacterium is a Bacillus.

Item 3.The method of item 1, wherein the bacterium is a Bacillus velezensis or a Bacillus amyloliquefaciens.

Item 4.The method of item 1, wherein the bacterium produces, or the biosurfactant-containing extract contains, at least 3 of bacilysin, difficidin, macrolactin H, bacillaene, bacillomycin D, fengycin, surfactin and bacillibactin.

Item 5.The method of any one of items 1-4, wherein the bacterium encodes at least 10 of SEQ ID NOs: 1-111.

Item 6.The method of any one of items 1-5, wherein the bacterium is a Bacillus velezensis OB9.

Item 7.The method of any one of items 1-6, wherein the bacterial or fungal pathogen is a Salmonella, an Escherichia coli, an Enterobacteriaceae, an Xanthomonas campestris, a Rhizoctonia solani or a Botrytis cinereal.

Item 8.The method of any one of items 1-6, wherein the bacterial pathogen is a Salmonella or a Xanthomonas campestris.

Item 9.The method of any one of items 1-8, wherein the bacterial or fungal pathogen is a food and/or plant pathogen.

Item 10. The method of any one of items 1-8, wherein the bacterial or fungal pathogen is a plant pathogen, and wherein the method comprises bacterizing a plant or coating food or a plant with the bacterium.

Item 11. The method of item 10, wherein the plant is a tomato plant or a lettuce plant.

Item 12. The method of any one of items 1-11, wherein the bacterial pathogen is a biofilm producing bacterial pathogen.

Item 13. The method of item 12, wherein the biofilm producing bacterial pathogen is a Salmonella.

Item 14. The method of item 13, wherein the biofilm producing bacterial pathogen is a Salmonella enterica.

Item 15. The method of any one of items 1-14, wherein the method uses the biosurfactant-containing extract isolated from said bacterium.

Item 16. The method of item 15, wherein the method uses an acid precipitate fraction of the biosurfactant-containing extract.

Item 17. A plant or plant part bacterized or coated with a Bacillus or a biosurfactant-containing extract isolated from the Bacillus.

Item 18. The plant or plant part of item 17, wherein the Bacillus is a Bacillus velezensis.

Item 19. The plant or plant part of item 17 or 18, wherein the Bacillus produces, or the biosurfactant-containing extract contains at least 3 of bacilysin, difficidin, macrolactin H, bacillaene, bacillomycin D, fengycin, surfactin and bacillibactin.

Item 20. The plant or plant part of any one of items 17-19, wherein the Bacillus encodes at least 10 of SEQ ID NOs: 1-111.

Item 21. The plant or plant part of any one of items 17-20, wherein the Bacillus is Bacillus velezensis OB9.

Item 22. The plant or plant part of any one of items 17-21, which is a tomato or a lettuce plant, plant part or cell.

Item 23. A method of protecting a plant or plant part against a bacterial or fungal pathogen comprising bacterizing or coating the plant or plant part with a Bacillus or a biosurfactant-containing extract isolated from the Bacillus, wherein the Bacillus produces or the extract contains at least 3 of bacilysin, difficidin, macrolactin H, bacillaene, bacillomycin D, fengycin, surfactin and bacillibactin.

Item 24. The method of item 23, wherein the Bacillus is a Bacillus velezensis.

Item 25. The method of item 23 or 24, wherein the Bacillus encodes at least 10 of SEQ ID NOs: 1-111.

Item 26. The method of one of items 23-25, wherein the Bacillus is a Bacillus methylotrophicus OB9.

Item 27. The method of any one of items 23-26, wherein the method comprising bacterizing or coating the plant.

Item 28. The method of any one of items 23-26, wherein the method comprising bacterizing or coating the plant part.

Item 29. The method of any one of items 23-28, wherein the bacterial or fungal pathogen is a Salmonella, an Escherichia coli, an Enterobacteriaceae, an Xanthomonas campestris, a Rhizoctonia solani or a Botrytis cinereal.

Item 30. The method of any one of items 23-29, wherein the bacterial pathogen is a Salmonella or a Xanthomonas campestris.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A-C. Distribution of bacterial genera in three oil wells. The numbers in each pie chart represent the isolates of each genus. The numbers in brackets represent the abundance of bacterial isolates of each genus relative to the total number of isolates recovered from each oil well. (FIG. 1A) Well 10-22, (FIG. 1B) Well 5-27, and (FIG. 1C) Well 10-17. Data represent the average of three-replicate samples.

FIGS. 2A-B. The antimicrobial activities of B. methylotrophicus OB9 (B.m.OB9) against plant and foodborne pathogens. (FIG. 2A) Confrontation co-culture assay with the fungal plant pathogen, Rhizoctonia solani. (FIG. 2B) Agar diffusion assays with Xanthomonas campestris B07.007 (X. campestris), Salmonella enterica subsp. enterica Newport SL1 (S. enterica), Escherichia coli E14-6 (E. coli). The (−ve) series denotes confluent growth of each organism on the control plates, while in the (+ve) series (Diffusion Agar Assay) the same organisms were challenged with B. methylotrophicus OB9 (as noted by the zone of clearing around B. methylotrophicus OB9).

FIGS. 3A-C. Thin layer chromatography (TLC) separation and biosurfactant assessment of cell free supernatant (CFS) and acid precipitate fraction (APF) extracts from B. methylotrophicus OB9.(FIG. 3A) TLC plate exposed to UV light with fraction numbers and Rf values denoted for CFS and APF preparations. The biosurfactant properties of TLC-isolated APF fractions of B. methylotrophicus OB9 were analyzed by the drop collapse assay (FIG. 3B) with APF fractions 1-5 and Triton™ X-100 (T) as a control and the oil spreading assay (FIG. 3C) with APF fractions 1-5, water (W) and Triton™ TM X-100 (T).

FIGS. 4A-E. Antimicrobial assessment of APF extracts from B. methylotrophicus OB9. (FIG. 4A) Antimicrobial activity of APF fractions F3 and F5 with the agar diffusion assay against Salmonella Typhimurium producing four biofilm morphotypes. From left to right: column 1 contains negative controls (-ve) for growth of each of the four strains (rows 1-4 are UMR1, MAE14, MAE299 and MAE775, respectively); columns 2-4 contain the same strains, but assayed against fraction 3 (F3), fraction 5 (F5) or bleach as positive control (+ve), respectively. FIGS. 4B-D: Additional diffusion assays of F3, F5, 1% (v/v) acetic acid, and 1% lactic acid against Salmonella agona PARC #5 (designated S. enterica strain A) (FIG. 4B), Salmonella serovar I:Rough-O:e,h:e,n (designated S. enterica strain B) (FIG. 4C) and E. coli E14-6 (E. coli) (FIG. 4D). In FIGS. 4B-D, upper panels show fractions F3 and F5 with the noted bacteria and the lower panels show fractions F3 and F5 with the noted bacteria and acetic acid or lactic acid. FIG. 4E shows the antagonistic effect of F3 and F5 against Rhizoctonia solani (top two panels), while the bottom two panels display negative control growth (−) and APF fraction 5 (F5) assayed Botrytis cinerea. Antimicrobial assessment of APF was conducted three times.

FIGS. 5A-D. Suppression of Salmonella and Xanthomonas growth in co-cultures with B. methylotrophicus OB9. FIGS. 5A-B: Temporal microbial counts of Salmonella enterica Newport (SL1) (FIG. 5A) and Bacillus methylotrophicus (B.m. OB9) (FIG. 5B) in single cultures and in co-cultures with OB9 over 72 hours. FIGS. 5B-C: Temporal microbial counts of Xanthomonas (FIG. 5C) and OB9 (FIG. 5D) in single cultures and in co-cultures with B. methylotrophicus OB9 over 72 hours.

FIGS. 6A-D. Suppression of Salmonella enterica subsp. enterica Newport SL1 (S. enterica) and Xanthomonas campestris B07.007 (X. campestris) growth in bacterized plants with Bacillus methylotrophicus OB9 (B.m. OB9). (FIG. 6A) Control treatment (tomato+S. enterica subsp. enterica Newport SL1 (S. enterica)) displays a tomato seedling in tissue culture infected with S. enterica. Recovery of S. enterica cells on culture medium from a detached leaf (shown) infected with S. enterica representative of CFU/gram tissue is depicted on the agar plate. (FIG. 6B) Treatment (Tomato+B.m. OB9+S. enterica) consisting of a tomato seedling in tissue culture bacterized with Bacillus methylotrophicus OB9 (B.m. OB9) and infected with S. enterica. Recovery of B.m. OB9 cells from a detached bacterized leaf (shown) infected with S. enterica (B.m. OB9+S. enterica) representative of CFU/gram tissue is depicted on the agar plate. (FIG. 6C) Control treatment (Lettuce+X. campestris) showing a lettuce seedling in tissue culture infected with X. campestris B07.007 and an infected detached leaf is shown. Recovery of X. campestris cells on culture medium from a detached leave (shown) infected with X. campestris representative of CFU/gram tissue is depicted on the agar plate. (FIG. 6D) Treatment (Lettuce+B.m.OB9+X. campestris) of a lettuce seedling in tissue culture bacterized with B.m. OB9 and infected with X. campestris B07.007. Recovery of B.m. OB9 cells but not X. campestris cells on culture medium from a detached bacterized leave (shown) infected with X. campestris representative of CFU/gram tissue is depicted on the agar plate.

FIGS. 7A-D. Quantification of cell numbers of B. methylotrophicus OB9 (B.m. OB9), Salmonella enterica subsp. enterica Newport SL1 (S. enterica) and Xanthomonas campestris B07.007 (X. campestris) recovered from leaf tissues. Leaf tissues bacterized with B.m.OB9 and challenged with S. enterica (FIG. 7A) and X. campestris (FIG. 7B). FIGS. 7C-D: Tomato and lettuce leaf samples that were coated with B.m. OB9 and infected with S. enterica (FIG. 7C) and X. campestris (FIG. 7D). (BI) and (CI) samples represent B.m. OB9bacterized (BI) or coated (CI) tissues that are infected with either S. enterica or X. campestris. (NBI) and (NCI) treatments represent non-bacterized and non-coated plant samples that were infected with S. enterica or X. campestris controls, respectively. Data represent the average of 5 replicates per treatment.

### DESCRIPTION OF ILLUSTRATIVE EMBODIMENTS

The present disclosure is illustrated in further details by the following non-limiting examples.

### EXAMPLE 1: MATERIALS AND METHODS

### Source of Crude Oil Samples and Organisms

Crude oil samples were obtained from the operating area of Talisman Energy Inc., which is located in Chauvin, Alberta. The inventors were provided with duplicate samples representing pooled samples from wells of each of the three batteries (battery 10-22, battery 5-27 and battery 10-17), using sterile 500 mL Schott bottles tightly sealed, kept on ice during transportation and stored at 4° C. for DNA extraction (Table II, below). The physical and chemical characteristics of the crude oil are listed in Table II. Target organisms, consisting of a panel of 41 bacterial and fungal strains (listed in Table III, below), were tested in dual diffusion assays with biosurfactant-producing strains. Salmonella enterica subsp. Enterica strains belonging to 17 different serovars, including top clinical serovars (e.g., Typhimurium, Heidelberg, Newport, Infantis, Thompson and Braenderup) and environmental samples, were classified according to the four Salmonella biofilm morphotypes displayed on congo red plates (Römling et al., 1998). Strains with the morphotypes: rdar (red dry and rough) are cellulose and curli positive, pdar (pink dry and rough) are cellulose positive, bdar (brown dry and rough) are curli positive and saw (smooth and white) are negative for both components.

TABLE II
Physical and chemical properties of oil samples
from oil batteries 10-22, 5-27, and 10-17

| | Location | | | | | |
|---|---|---|---|---|---|---|
| Properties | Chauvin, Alberta | | Chauvin, Alberta | | Chauvin, Alberta | |
| Oil Battery | 10-22 | | 5-27 | | 10-27 | |
| Number of | 36 | | 119 | | 162 | |
| wells^{a} | | | | | | |
| pH | 7.4 | | 7.4 | | 7.4 | |
| Specific gravity | 1.06 | kg/m³ | 1.06 | kg/m³ | 1.06 | kg/m³ |
| Water content | 0.40% | | 0.29% | | 0.14% | |
| Absolute | 955.1 | kg/m³ | 917 | kg/m³ | 946.7 | kg/m³ |
| density | | | | | | |
| API gravity | 16.6 | kg/m³ | 22.8 | kg/m³ | 17.9 | kg/m³ |
| Sulphur | 25.3 | g/kg | 24.7 | | 28.7 | g/kg |
| Total solids | 81315 | mg/lt | 82900 | mg/lt | 86159 | mg/lt |
| dissolved | | | | | | |
| Viscosity | 863 | mPas | 87.05 | mPas | 644.5 | mPas |

^{a}Recovery and isolation of microbes were pooled from duplicate oil samples provided from each battery.

TABLE III
Source of organisms

| | | | | | |
|---|---|---|---|---|---|
| | | | Biofilm | | |
| | | | formation | | |
| Number | Strain ID | Identity | at 28° C. | Origin/morphotype | Source/provider |

BACTERIA
Salmonella strains/morphotypes

| | | | | | |
|---|---|---|---|---|---|
| 1 | UMR1 | Salmonella typhimurium | rdar* | Control | 1 |
| 2 | MAE14 | S. typhimurium | pdar* | Control | 1 |
| 3 | MAE299 | S. typhimurium | bdar* | Control | 1 |
| 4 | MAE775 | S. typhimurium | baw* | Control | 1 |
| 5 | S1V1 | Salmonella sp. | pdar | Environment | 1 |
| 6 | S3PP | Salmonella sp. | saw | Environment | 1 |
| 7 | S13V2 | Salmonella sp. | rdar | Environment | 1 |
| 8 | S2V3 | Salmonella sp. | rdar & saw | Environment | 1 |
| 9 | S1V3 | Salmonella sp. | saw | Environment | 1 |
| 10 | S4V2 | Salmonella sp. | pdar & saw | Environment | 1 |
| 11 | S10V1 | Salmonella sp. | pdar | Environment | 1 |
| 12 | S12PP | Salmonella sp. | rdar | Environment | 1 |
| 13 | S18V2 | Salmonella sp. | pdar & saw | Environment | 1 |
| 14 | S2V2 | Salmonella sp. | Combination | Environment | 1 |
| 15 | SC01 | Salmonella Serotype | rdar | Environment | 1 |
| | | I: 4, 5, 12: b: | | | |
| 16 | SC16 | Salmonella Serotype | saw | Environment | 1 |
| | | I: Rough-O:: e, n, x | | | |
| 17 | SC04 | Salmonella Serotype | rdar | Environment | 1 |
| | | Braenderup | | | |
| 18 | SCS7 | Salmonella Serotype | saw | Environment | 1 |
| | | Typhimurium | | | |
| 19 | SC110 | Salmonella Serotype | rdar | Environment | 1 |
| | | I: RoughO: y: e, n, x | | | |
| 20 | SC12 | Salmonella Serotype | rdar | Environment | 1 |
| | | I: Rough-O: e, h | | | |
| 21 | SC18 | Salmonella Serotype | rdar | Environment | 1 |
| | | Hartford | | | |
| 22 | SCS2 | Salmonella | rdar | Environment | 1 |
| | | Serotype: RoughO: e, h: e, | | | |
| 23 | WTCR5 | Salmonella Serotype | rdar/saw | Environment | 1 |
| | | I: 6, 7: r | | | |
| 24 | WTCR6 | Salmonella Serotype | rdar/saw | Environment | 1 |
| | | Stanley | | | |
| 25 | WTCR9 | Salmonella Serotype | rdar/saw | Environment | 1 |
| | | Infantis | | | |
| 26 | WTCR30 | Salmonella Serotype- | rdar/saw | Environment | 1 |
| | | Schwarzengrund | | | |
| 27 | WTCR22 | Salmonella Serotype | rdar/saw | Environment | 1 |
| | | Thompson | | | |
| 28 | WTC27 | Salmonella serotype | saw | Environment | 1 |
| | | Heidleberg | | | |
| 29 | WTC28 | Salmonella Serotype | rdar/saw | Environment | 1 |
| | | Monschaui | | | |
| 30 | WTCT4 | Serotype Heidleberg | rdar/saw | Environment | 1 |
| 31 | PARC#5 | S. agona | rdar/saw | Mung bean | 3 |
| 32 | SL1 | S. Newport | rdar | Human gut | 2 |
| 33 | SL2 | S. Hartford | rdar | Human gut | 2 |

Escherichia species

| | | | | | |
|---|---|---|---|---|---|
| 34 | E3-6 | Escherichia coli | NA | NA | 1 |
| 35 | E10-6 | E. coli | NA | NA | 1 |
| 36 | E14-6 | E. coli | NA | NA | 1 |
| 37 | E15-6 | E. coli | NA | NA | 1 |

Xanthomonas sp.

| | | | | | |
|---|---|---|---|---|---|
| 38 | B07.007 | Xanthomonas campestris | rdar | Lettuce | 4 |
| | | pv. vitians | | | |

FUNGI
Rhizoctonia sp.

| | | | | | |
|---|---|---|---|---|---|
| 39 | AG3-114 | R. solani | NA | Potato | 5 |
| 40 | AG1-1-ROS- | R. solani | NA | Soybean | 6 |
| | 2A4) | | | | |

Botrytis

| | | | | | |
|---|---|---|---|---|---|
| 41 | F-014 | Botrytis cinerea | NA | Strawberry | 7 |

*Strains displaying rdar (red dry and rough) are cellulose and curli positive, strains displaying pdar (pink dry and rough) are cellulose positive, strains displaying bdar (brown dry and rough) are curli positive, and strains displaying saw (negative for both components) are negative for both components, NA: not applicable.
1. J. Weadge, Wilfrid Laurier University, Ontario; U. Romling et al., (2003). Int. J. Med. Microbiol. 293, 273-285.
2. S. Bekal, The Laboratory of Public Health of Quebec (QPHL), Quebec.
3. S. Orban, Agriculture and Agri-Food Canada (AAFC), BC.
4. V. Toussaint, AAFC, St. Jean sur Richelieu, Quebec.
5. M. A. Cubeta , N.C. States University, NO, USA.
6. P. Ceresini, UNESP Sao Paolo, Brazil.
7. S. Jabaji, McGill University, Quebec.

### Isolation of Oil-Dwelling Bacteria

Unless otherwise stated, manipulation of oil samples was performed under sterile conditions in the laminar flow hood in order to avoid any contamination. Crude oil samples (10 mL of each) were mixed with an equal volume of sterile distilled water. The emulsion was vortexed and then continuously agitated (2×g) on a rotary shaker for 2 h, before the aqueous phase was recovered with a sterile pipette. This procedure was repeated five times, collecting a total of 50 mL of the aqueous phase for each sample from each well. Aliquots (100 μL) of the aqueous phase were plated on different microbiological culture media; potato dextrose agar (PDA), malt extract agar (MEA), nutrient agar (NA) and lysogeny broth agar (LBA) (BDH chemical Ltd, Mississauga, ON, Canada), and incubated at 28° C. Bacterial colonies that grew on culture media were passed through four rounds of single colony isolation by streaking them on the above-mentioned culture media to ensure purity of each strain prior to long-term storage. Pure bacterial cultures were stored in 20% (v/v) glycerol and kept at −80° C. until further use.

### Molecular Identification of Bacteria Recovered from Oil Bacterial strains were grown in LB broth for 18 h with agitation to obtain a final concentration of 10⁸-10¹⁰ colony forming units (CFU) mL⁻¹. Cells were pelleted by centrifugation, and DNA was extracted using the DNeasy® Blood & Tissue kit (Qiagen, Mississauga, Ontario) following the manufacturer's instructions. DNA concentration and quality were confirmed spectrophotometrically with a NanoDrop™ ND1000 spectrophotometer (NanoDrop, Wilmington, Del. USA) and on a 1% (w/v) agarose gel, respectively. The 16S rRNA gene sequences were amplified using the ITS primer pair 27F/534R (5′-AGAGTTTGATCCTGGCTCAG-3′) (SEQ ID NO: 112) and 534R (5′-ATTACCGCGGCTGCTGG-3′) (SEQ ID NO: 113) according to previously published protocols (Gagne-Bourgue et al., 2013; Scott et al., 2018) to putatively identify 123 bacterial strains. PCR amplification was performed in a Bio-Rad™ T100 Thermal Cycler using the iProof™ High-Fidelity (HF) PCR kit (Bio-Rad, Ontario, Canada) using 40 ng of genomic DNA for a 50 μL reaction following previously published protocols (Gagne-Bourgue et al., 2013; Scott et al., 2018). Negative and positive controls were included concurrently with each reaction according to previous protocols (Scott et al., 2018). Amplification of PCR products was confirmed on a 1% (w/v) agarose gel. Lower molecular weight PCR products were cloned into the pDrive™ vector (Qiagen) following the manufacturer's protocol. Purified plasmid DNA and PCR products were sequenced at Genome Quebec sequencing services (McGill University, Montreal, QC, Canada). Sequences were subjected to Blast™ search against the NCBI database using the algorithm megablast (http://www.ncbi.nlm.nih.gov/blast) to confirm identity through sequence homology. The obtained sequences were submitted NCBI GenBank.

### Enrichment of Biosurfactant Production by Oil-Dwelling Bacteria

For efficient degradation of complex hydrocarbon oil and the production of biosurfactants, 3.27 g/L of Bushnell and Haas (BHM) (BDH chemical Ltd) supplemented with 2% of each of crude oil (v/v), glucose (w/v) and molasses (v/v) as sole sources of carbon, adjusted to pH 7.0 and sterilized at 21 psi for 20 min was used. A 1 mL volume of bacterial cultures (grown at 22° C. for 18-24 h with agitation in LB broth) with an OD₆₀₀ between 0.6 and 1.0 was transferred to 100 mL of the carbon-amended BHM media. Inoculated media was incubated with continuous agitation (2×g) at 30° C. for 7 d and then the cell-free supernatant (CFS) was collected by centrifugation (6500×g at 4° C. for 20 min). Removal of any residual oil and/or live bacterial cells present in the cell-free supernatant was accomplished by filtration using a 0.22 μm Millipore™ filter (Millipore Sigma, Ontario, Canada) and kept at 4° C. until further use.

### Assays for Biosurfactant Production

Bacterial isolates originating from oil batteries (10-22, 5-27 and 10-17) were screened for biosurfactant production by applying the most commonly used assays in the literature; the oil-spreading test and drop-collapse assay (Thavasi, 2011; Youssef et al., 2004). Isolates were considered to have significant biosurfactant production if the clearing zone they produced was at least ≤1.0 cm in diameter in the oil spreading assay and ≤3.0 mm in the drop collapse assay (Youssef et al., 2004). In all assays unless otherwise stated, Triton™ X-100 (10 mg/mL) was used as the positive control, while sterile de-ionized water and un-inoculated hydrocarbon-amended BHM medium served as negative controls. All tests were replicated twice for each bacterial strain tested. Based on the above-mentioned criteria, the top biosurfactant producers were further screened using the CTAB agar method, emulsification assay, microplate assay, and hemolytic assay.

Drop collapse assay. The wells of a polystyrene 96 well micro-plate lid (Corning incorporated, USA) were coated with 2 μL of crude oil and left to dry for 24 h at 22° C. Filtered cell-free supernatant (5 μL) was transferred to the center of the oil coated well. The results were recorded after 1-2 min and considered positive for biosurfactant production when the oil drop was flat. Those that gave rounded drops were scored negative, an indication of the absence of biosurfactant production (Jain et al., 1991).

Oil spreading assay. The oil-spreading assay was performed in polystyrene petri dishes (100 mm×15mm) containing 20 μL of crude oil that was carefully layered over 20 mL of distilled water. A drop (˜10 μL) of filtered supernatant was carefully pipetted onto the center of the oil layer. The diameter of the clear zone on the surface of the oil layer was measured and compared to the negative controls (Ibrahim et al., 2013).

CTAB agar assay. CTAB agar plates were prepared by adding 0.15 g of cetyltrimethylammonium bromide (CT-Aldrich, Oakville, Ontario, Canada), 0.005 g Methylene blue (Sigma-Aldrich) and 12 g of agar to 1 L of distilled water, adjusted to pH 7 and sterilized (Tahzibi et al., 2004). Two holes (6.5 mm diameter) were made in the CTAB plates, and approximately 150 μL of filtered cell-free supernatant was loaded inside each hole. Plates were incubated at 37 ° C. for 48 h. Cell free supernatant containing anionic surfactant produced blue halos around the wells in which they were placed. The diameter of the halo was measured and compared with positive and negative controls.

Emulsification assay. A volume of 1 mL of the cell-free supernatant was added to 5 mL of 50 mM Tris buffer (pH 8.0) in a 30 mL screw-capped test tube. Crude oil was tested for emulsification activity. Crude oil (5 mg) was added to both layers and vortexed for 1 min and then the emulsion mixture was allowed to settle for 20 min. The optical density of the emulsified mixture was measured at 610 nm (Muthezhilan et al., 2014). A negative control consisted of only buffer solution and crude oil with Triton™ X-100 was used as the positive control.

Microplate assay. An aliquot of 50 μL of filtered cell-free supernatant was placed in domed PCR caps (Ultident Scientific, St. Laurent, Quebec, Canada) that were oriented over a grid of 1 mm×1 mm squares. The presence of biosurfactant was confirmed by the distortion of the grid image and were qualitatively compared to the positive and negative controls (Walter et al., 2010).

Hemolytic activity. Blood agar plates were prepared by adding 5% (v/v) of sheep blood (Fisher scientific) to a sterilized mixture of NaCl (10 g), yeast (5 g), tryptone (10 g) and agar (15 g) in 1 L of distilled water (Phan et al., 2013). Approximately 150 μL of filtered cell free supernatant of each bacterial isolate was loaded into each well (6.5 mm in diameter) made by a cork borer in the blood agar plates and incubated at 30° C. for 24-48 h. Biosurfactant biosynthesis was confirmed by hemolysis activity as indicated by the presence of clearing zones around the wells. The diameter of the lysis zone was scored as ‘−’ no lysis, ‘+’ partial hemolysis, ‘++’ moderate hemolysis , ‘+++’ complete hemolysis.

Antimicrobial Activity of Whole Cultures of B. methylotrophicus OB9

Isolate OB9 exhibited the highest activity for oil displacement and emulsification activity. This strain was identified as B. methylotrophicus based on sequencing data and hereby designated as B. methylotrophicus OB9 (Jeukens et al., 2015). This strain was further screened for its antimicrobial activity against a wide panel of clinical and environmental bacterial strains (Table V) employing the Burkholder agar diffusion assay (Burkholder et al., 1944), and also against fungal phytopathogens using the dual confrontation assay (Gagne-Bourgue et al., 2013).

TABLE IV
Top biosurfactant-producing isolates of three oil wells 10-22, 5-27, 10-17

| | | | OIL | DROP | GENBANK |
|---|---|---|---|---|---|
| | | STRAIN | SPREADING | COLLAPSE | ACCESSION |
| RANK | NAME | ID | (CM)* | (MM)** | NUMBER |
| Well 10-52 | | | | | |
| 1 | Bradyrhizobium sp. | OB55 | 1.6 ± 0.1& | 3.5 ± 0.0 | MG926629 |
| 2 | Streptomyces sp. | OB45 | 1.6 ± 0.7 | 3.0 ± 0.0 | MG926620 |
| 3 | Streptomyces sp. | OB44 | 2.0 ± 0.6 | 3.5 ± 0.0 | MG926618 |
| 4 | Bacillus amyloliquefaciens | OB10 | 2.1 ± 0.2 | 3.5 ± 0.0 | MG926639 |
| 5 | Bacillus nealsonii | OB51 | 2.6 ± 0.0 | 4.0 ± 0.0 | MG926626 |
| 6 | Microbacterium sp. | OB14 | 2.3 ± 0.2 | 3.5 ± 0.0 | MG926642 |
| 7 | Bacillus methylotrophicus | OB43 | 2.4 ± 0.7 | 3.5 ± 0.0 | MG926614 |
| 8 | Streptomyces yanglinensis | OB41 | 2.6 ± 0.1 | 3.5 ± 0.0 | MG926610 |
| 9 | Bacillus amyloliquefaciens | OB5 | 3.2 ± 0.3 | 3.8 ± 0.3 | MG924914 |
| 10 | Streptomyces sp. | OB42-1 | 3.3 ± 0.1 | 3.5 ± 0.1 | MG926612 |
| 11 | Bacillus amyloliquefaciens | OB6 | 3.6 ± 0.3 | 4.3 ± 0.3 | MG926633 |
| 12 | Bacillus velezensis | OB9 | 3.8 ± 0.1 | 4.3 ± 0.6 | MG926638 |
| | (previously methylotrophicus) | | | | |
| Well5-27 | | | | | |
| 1 | Bacillus simplex | E05 | 1.5 ± 0.0 | 3.5 ± 0.0 | MG946778 |
| 2 | Micrococcus testaceum | E47 | 1.5 ± 0.2 | 3.3 ± 0.1 | MG926596 |
| 3 | Rhodococcus sp. | E51-2 | 1.6 ± 2.7 | 3.5 ± 0.0 | MG627949 |
| 4 | Bacillus methylotrophicus | E62 | 1.7 ± 0.3 | 3.7 ± 0.2 | MG946778 |
| 5 | Pseudomonas sp. | E14 | 2.2 ± 0.1 | 4.8 ± 0.3 | MG946770 |
| 6 | Bacillus subtilis | E64 | 2.0 ± 0.2 | 3.8 ± 0.3 | MG946784 |
| 7 | Bacillus subtilis | E68 | 2.6 ± 0.3 | 3.8 ± 0.0 | MG946779 |
| 8 | Arthrobacter sp. | E45 | 2.9 ± 0.4 | 4.2 ± 0.3 | — |
| Well 10-17 | | | | | |
| 1 | Bacillus methylotrophicus | M16-3 | 2.2 ± 0.0 | 3.4 ± 0.1 | MH627955 |
| 2 | Bacillus subtilis | M46-2 | 2.6 ± 0.1 | 3.3 ± 0.1 | MH627971 |
| 3 | Bacillus methylotrophicus | M05-2 | 2.7 ± 0.2 | 3.4 ± 0.1 | MH627945 |
| 4 | Bacillus subtilis | M43 | 2.9 ± 0.1 | 3.4 ± 0.2 | MH627966 |
| 5 | Bacillus amyloliquefaciens | M36 | 2.9 ± 0.1 | 3.4 ± 0.2 | MH627961 |
| 6 | Bacillus amyloliquefaciens | M27 | 3.0 ± 0.1 | 3.2 ± 0.1 | MH627959 |
| 7 | Bacillus velezensis | M50 | 3.0 ± 0.1 | 3.4 ± 0.1 | MH627972 |
| 8 | Kokuria rhizophila | M20 | 3.1 ± 0.1 | 3.5 ± 0.0 | MH627956 |
| 9 | Staphylococcus saprophyticus | M44 | 3.1 ± 0.0 | 3.2 ± 0.1 | MH627967 |
| 10 | Bacillus velezensis | M05 | 3.2 ± 0.2 | 3.4 ± 0.1 | MH627944 |
| 11 | Bacillus amyloliquefaciens | M30 | 3.4 ± 0.1 | 3.4 ± 0.1 | MH627960 |
| 12 | Bacillus velezensis | M09 | 3.4 ± 0.0 | 3.4 ± 0.2 | MH627948 |
| 13 | Bacillus velezensis | M21 | 3.4 ± 0.1 | 3.2 ± 0.1 | MH627957 |
| 14 | Bacillus subtilis | M42 | 3.5 ± 0.0 | 3.2 ± 0.1 | MH627965 |

*For oil spreading assay clearing zone of ≥1.5 cm is considered positive.
**For drop collapse assay above ≥3 mm is considered positive
&Values represent the mean of three replicates ± standard error of the mean (S.E.)

Agar diffusion assay. All target bacterial strains and B. methylotrophicus OB9 were grown in LB broth at 27° C. for 16-18 h with constant shaking to achieve mid-log phase, with a cell density of 10⁶ CFU mL⁻¹ as assessed based on standard curves relating optical density at 600 nm (OD₆₀₀) to plate counts on LBA plates. Cells were pelleted by centrifugation (6500×g at 4° C. for 20 min. and suspended in sterile ddH₂O. A total volume of 30 μL of each bacterial suspension was mixed gently with 2.5 mL of molten half-strength LB agar and poured into culture plates. An aliquot (10 μL) of B. methylotrophicus OB9 was spotted in the center of the bacterial lawn and plates were incubated at 24° C. Zones of bacterial growth-inhibition subjacent to the spotted B. methylotrophicus OB9 inoculum were recorded after 24-48 h and were found to vary in diameter between 3 to 6 mm depending on the potency of B. methylotrophicus OB9 antimicrobials that diffused into the agar. Individual trials were performed in triplicate and the entire assay was repeated twice to confirm the findings.

Dual confrontation assay. Confrontation assay plates were used to screen B. methylotrophicus OB9 for its ability to inhibit radial growth of Botrytis cinerea, and Rhizoctonia solani isolates according to the modified method of Gagné-Bourgue (Gagne-Bourgue et al., 2013). PDA plates were inoculated in the center with a 5 mm diameter mycelial plug taken from the edge of an actively growing fungal colony. A 5 μL volume of B. methylotrophicus OB9 (10⁶ CFU mL⁻¹) was deposited 25 mm on either side of the fungal colony. Triplicate plate assays were performed for each target fungal organism and radial growth inhibition of the fungus was measured 5 d post confrontation.

Purification and Fractionation of Biosurfactants of B. methylotrophicus OB9 by Thin Layer Chromatography (TLC)

The biosurfactant-producing B. methylotrophicus OB9 was cultured in 2 L of BHM broth supplemented with 2% (w/v) glucose, 2% (v/v) molasses and 2% (v/v) oil for 7 d with agitation (2×g) at 30° C. Bacterial cells were removed by centrifugation (5000×g at 4° C.) and the cell—free supernatant (CFS) was acidified to pH 2.0 with 2N HCL at 4° C. for 16 h resulting in a precipitate that was collected by centrifugation (5000×g for 10 min at 4° C.). The acid precipitate fraction (APF) was dissolved in water (100 mg/mL) and adjusted to pH 7.0 using 1N NaOH. The CFS sample and clean BHM culture medium were concentrated by freeze-drying for 48 h. All samples were stored at −80° C. until further use.

Separation of CFS and APF fractions was performed by thin layer chromatography (TLC). Acid-cleaned glass plates (20×20 cm) were coated with a thin layer of silica emulsion (160 grams of silica with 15% (w/v) calcium sulphate and with fluorescent indicator GF254 dissolved in 500 mL of double distilled water) purchased from Sigma-Aldrich and dried for 16-18 h at 22° C. Activation of silica plates was done by heating the TLC plates at 120° C. for 30 min prior to use. An amount of 100 mg of each of CFS and APF were dissolved in 400 μL of chloroform: methanol (2:1, v/v) and separated on activated TLC plates with chloroform: methanol: water (70:26:4) as the developing solvent system. Plates were observed under UV (λ=254 nm) and the retention factor (Rf) values were calculated as distance travelled by the samples over the distance travelled by solvent. Only APF separated fractions were used for bioactivity assays. Bands corresponding to the desired Rf values were scraped and collected for extraction. Bands of the same Rf value from different plates were pooled together and extracted twice with chloroform: methanol (v/v/; 2:1). The collected fractions were vacuum-concentrated for 4-6 h at −60° C. using a Speed Vac™ v78100 (Labconco Corp, Kansas City Mo., USA), re-dissolved in 50 μL of methanol and tested against target bacteria and fungi, as well as for biosurfactant activity. Methanol (50 μL) was used as a negative control.

### Biosurfactant and Antimicrobial Activities of TLC Fractions

The biosurfactant activity of the fractions was tested using the oil-spreading and the drop collapse methods, as described above. Fractions were also tested for their antimicrobial activities against a number of bacteria and fungi using the Burkholder diffusion plate method as noted above. Briefly, 10 μL of the dissolved fractions were spotted in the middle of the plates. Positive controls consisted of 10 μL of bleach and of each of the following organic acids: 1% (v/v) of acetic acid, formic acid, citric acid and lactic acid. All plates were incubated at 24° C. and a clearing zone of 3 mm or greater after 24 h of incubation was considered positive for inhibition of growth. Individual trials were performed in triplicate and the entire assay was repeated twice to confirm the findings.

Interaction of B. methylotrophicus OB9 with S. enterica Newport SL1 and X. campestris B07.007 in Liquid Co-Culture

This method was performed to study how B. methylotrophicus OB9 directly interacts with target strains of Salmonella enterica Newport SL1 and X. campestris B07.007. Each bacterium was grown in LB broth until a cell density of 10⁶ CFU mL⁻¹ was achieved, as assessed based on standard curves relating optical density at 600 nm (OD₆₀₀) to plate counts on LBA plates. An equal volume of B. methylotrophicus OB9 culture and target organisms (Salmonella or Xanthomonas) was added to fresh LB liquid culture amended with 3% (w/v) yeast extract. Pure cultures of Salmonella enterica Newport SL1, X. campestris and B. methylotrophicus OB9 grown in amended LB broth served as positive controls. Liquid co-cultures and controls were incubated with agitation (2×g) at 28° C. To avoid changes in growth during co-culturing due to pH variation or nutrient limitations, every 10 h liquid co-cultures and controls were subjected to centrifugation (15 min at 5000×g) and bacterial pellets were suspended in fresh amended LB medium. Serial dilutions (10⁻³ to 10⁻⁵) from control and liquid co-cultures were plated on LBA after 3, 6, 12, 24 and 72 h of incubation, estimated as log₁₀ CFU mL⁻¹ and compared to the control treatments. There were three replicates per target organism and treatment. Individual trials were also repeated at least twice.

Bacterization and Internalization of B. methylotrophicus OB9 in Tissue Cultured Tomato and Lettuce Seedlings.

Vegetable cultivars. Organic Tomato (cv. Beefsteak) and Cos lettuce (cv. Parris Island) seeds (Vesey seed Ltd, York, PE, Canada) were surface sterilized using 70% (v/v) ethanol and 1.3% (v/v) sodium hypochlorite in a stepwise procedure according to Scott et al., 2018. The seeds were transferred to tissue culture tubes (25 mm×150 mm; VWR, PA, USA) containing 10 mL of Murashige and Skoog (MS) medium supplemented with 3% (w/v) sucrose (Gagné-Bourque et al., 2015). Tissue culture tubes were incubated under 200 μmol m⁻² s⁻¹ white fluorescent light, 18/6-h photoperiod, and 24° C. day/night temperature for 2 weeks.

Preparation of B. methylotrophicus 0B9 inoculum. Inoculum of B. methylotrophicus OB9 was prepared by transferring a single colony to 100 mL of LB broth and incubating with agitation (2×g) at 28° C. Bacterial cells were harvested by centrifugation (4500×g for 15 min at 4° C.), washed with10 mM phosphate buffer (PBS) containing 0.8% (w/v) NaCl, pH 6.5 (phosphate buffered saline or PBS) and then suspended in the same buffer following another round of centrifugation (Pillay et al., 1997). The density of the B. methylotrophicus OB9 suspension was adjusted to 10⁶ CFU mL⁻¹ as described above.

Bacterization of plant seedlings with B. methylotrophicus OB9. A 500 μL volume of B. methylotrophicus OB9 in PBS buffer was carefully dispensed on the surface of the MS media and close to the roots of 2-week-old tomato and lettuce seedlings. Non-bacterized seedlings received 500 μL of PBS only and served as negative controls. Inoculated and control seedlings were returned to the environmental growth chamber and grown for 2 more weeks. The experimental unit was an individual plant contained in a test tube (a replicate) and 5 replicates per treatment were evaluated in two separate experimental trials. Bacterial populations were transformed to obtain homogeneity of variances and expressed as log₁₀ (CFU per leaf fresh weight).

Colonization, internalization and retrieval of B. methylotrophicus OB9 from plant tissue. Recovery and colonization of plant tissues by B. methylotrophicus OB9 was confirmed by culture-dependent (cell count) and culture-independent (PCR assay) methods. Cell counts (CFU mL⁻¹) were estimated 2 weeks post inoculation (wpi). Root and leaf tissues (100 mg of each) of tomato and lettuce seedlings were surface sterilized as described above to ensure that only B. methylotrophicus OB9 that had internally colonized plant tissues remained. The efficiency of the sterilization procedure was tested using the imprint method and absence of growth on the imprinted culture medium indicated that the surface sterilization procedure was effective. Surface sterilized tissues (100 mg) were ground in sterile distilled water, serially diluted (10⁻¹ to 10⁻⁷) and spread on LBA plates to assess the presence of B. methylotrophicus OB9. The presence of B. methylotrophicus OB9 inside plant tissues was also confirmed by PCR amplification. Briefly, bacterized Plant DNA (200 mg) was reduced to powder in liquid nitrogen and genomic DNA was extracted using the CTAB extraction method (Huang et al., 2011; Porebski et al., 1997) that targeted the bacterial DNA in the plants. Genomic DNA from pure colonies of B. methylotrophicus OB9 was extracted and PCR was performed (Huang et al., 2011). The presence of B. methylotrophicus OB9 DNA in plant tissue was detected using specific primers [5′-CAAGTGCCGTTCAAATAG-3′ (SEQ ID NO: 114) (Forward) and 5′CTCTAGGATTGTCAGAGG-3′ (SEQ ID NO: 115) (reverse)] in 25 μL PCR reactions using 20 ng of DNA. The amplification and PCR conditions are described in detail in (Gagné-Bourque et al., 2015).

Protection of Tomato and Lettuce Leaves by B. methylotrophicus OB9 from Salmonella and Xanthomonas

Preparation of Salmonella and Xanthomonas for leaf inoculation. Salmonella enterica Newport SL1 (isolated from human gut) and X. campestris B07.007 (isolated from lettuce) were prepared by separately transferring a single colony of each strain into 100 mL of LB broth and incubating with agitation (2×g) at 28° C. Bacterial cells were harvested, and the density of each culture was adjusted to 10⁶ cells mL⁻¹ following the same procedures described for B. methylotrophicus OB9 cells above. Two separate experiments were set up to study the effect of B. methylotrophicus OB9 on growth of Salmonella and Xanthomonas on plants.

### Statistical Analysis

In the co-culture experiment, there were three replicates per target organism and treatment, and the assays were repeated twice. In the tissue culture experiments (experiment 1 and experiment 2), each experimental unit consisted of one leaf per petri plate (one replicate). There were 5 replicates per treatment, and the entire experiment was repeated twice. Data of bacterial cell counts in co-culture and in tissue-culture experiments (Experiments 1 and 2) were analyzed by One-way ANOVA using the JMP 13.0 software. All experimental data were tested for statistical significance using Tukey's HSD (P≤0.05).

### EXAMPLE 2: BACTERIAL DIVERSITY OF CRUDE OIL-INHABITING BACTERIA

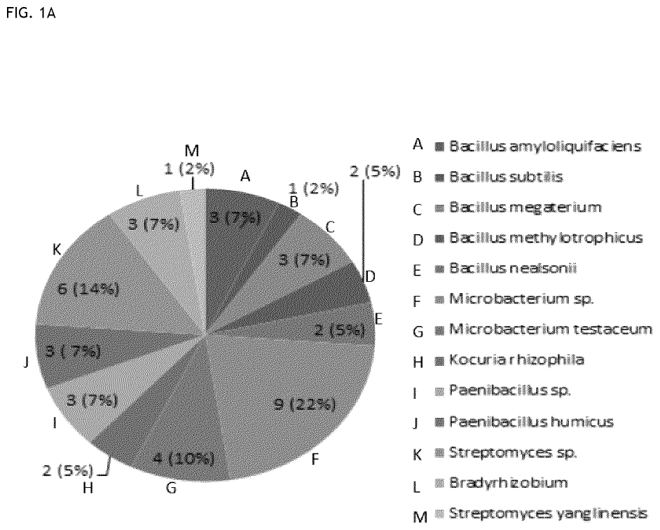

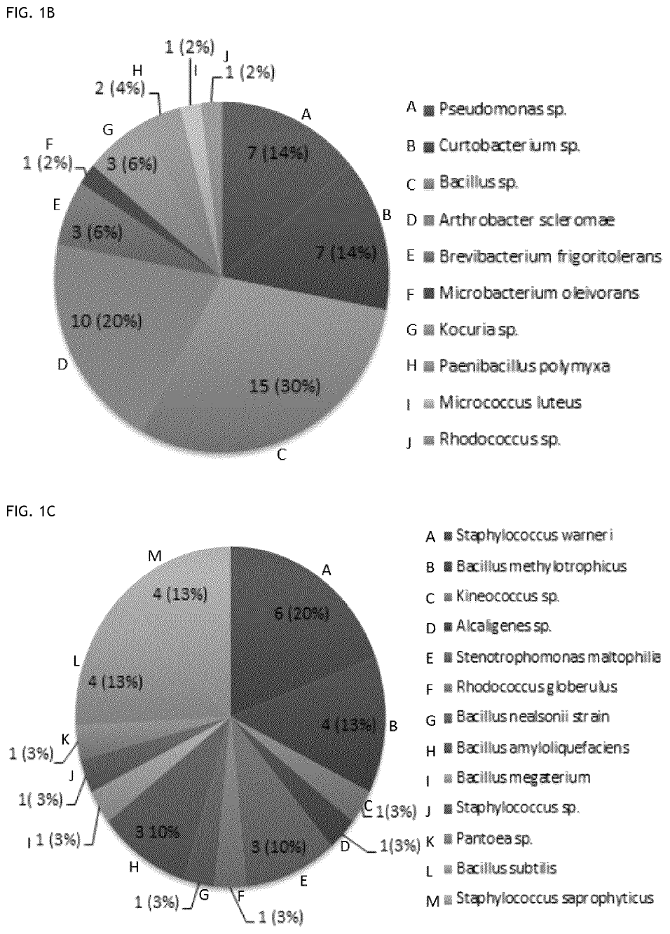

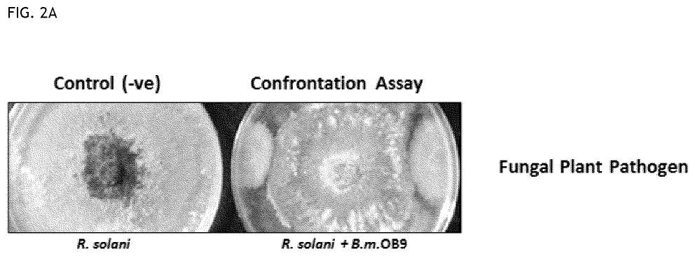

A total of 123 culturable bacteria, originating from crude oil samples were isolated from 3 oil batteries. A snapshot of the bacterial species in each oil battery, based on 16S rRNA gene sequences, indicated that the oil-inhabiting bacterial community is diverse (FIGS. 1A-C) and composed predominantly of bacteria (the majority identified to the species level) from 3 phyla (Actinobacteria, Proteobacteria, and Firmicutes) along with 17 discrete genera that shared high homology with known sequences. The full sequences of all strains have been deposited to GenBank under the following accession numbers (battery 10-22 MG924907-MG924915 and MG926585-MG92664, battery 10-17 MH627942-MH627972, and battery 5-27 MG946770-MG946789, and MG951760-MG951768). Strains belonging to the Gram-positive Bacillus genera (42, 39 and 32% from battery 10-17, 10-22, and 5-27, respectively) were the most common isolated across the three oil wells (FIGS. 1A-C). A total of 42 strains recovered from oil battery 10-22 were distributed among six different genera with the highest relative abundance among isolates being Bacillus (11/42), Microbacterium (13/42) and Streptomyces (7/42) species (FIG. 1A). Fifty isolates were recovered from oil battery 5-27 and belonged to 10 different genera, with Bacillus isolates again having the highest relative abundance (15/50). This battery also had isolates of Arthrobacter (10/50), Pseudomonas (7/50), Curtobacterium (7/50) and Brevibacterium (3/50) that were retrieved only from this oil well (FIG. 1B). Lastly, there were 31 isolates representing seven different genera recovered from well 10-27, with isolates of Staphylococcus (11/31), Stenotrophomonas (3/31), Alcaligenes (1/31), Kineococcus 1(31) and Pantoea (1/31) associated exclusively with this well (FIG. 1C).

### EXAMPLE 3: BIOSURFACTANT PRODUCTION

Thirty-four (34) isolates associated with Bacillus, Streptomyces, Microbacterium, Micrococcus, Rhodococcus, Pseudomonas, Arthrobacter and Staphylococcus genera retrieved from the three oil wells were identified as biosurfactant-producing bacteria using the oil—spreading and the drop collapse methods (Table IV). Isolates of Bacillus species showed the highest biosurfactant activities based on both tests. Strain OB9 isolated from oil well 10-22 and identified as B. methylotrophicus by whole genome sequencing (Jeukens et al., 2015, nucleotide sequence deposited at DDBJ/EMBUGenBank under accession number LGAU00000000) exhibited the highest biosurfactant activity followed by four strains of B. amyloliquifaciens (strains OB5, OB6, M30 and M09). Furthermore, these strains also tested positive for biosurfactant activity using the blood agar lysis method (a zone of 2.5 cm or greater), the CTAB method (a clearing zone of more than 1 mm), a significant grid using the microplate assay and in the emulsification assays (OD₆₀₀ value of 1.0) (Data not shown).

### EXAMPLE 4: ANTIMICROBIAL ACTIVITY OF B. METHYLOTROPHICUS OB9

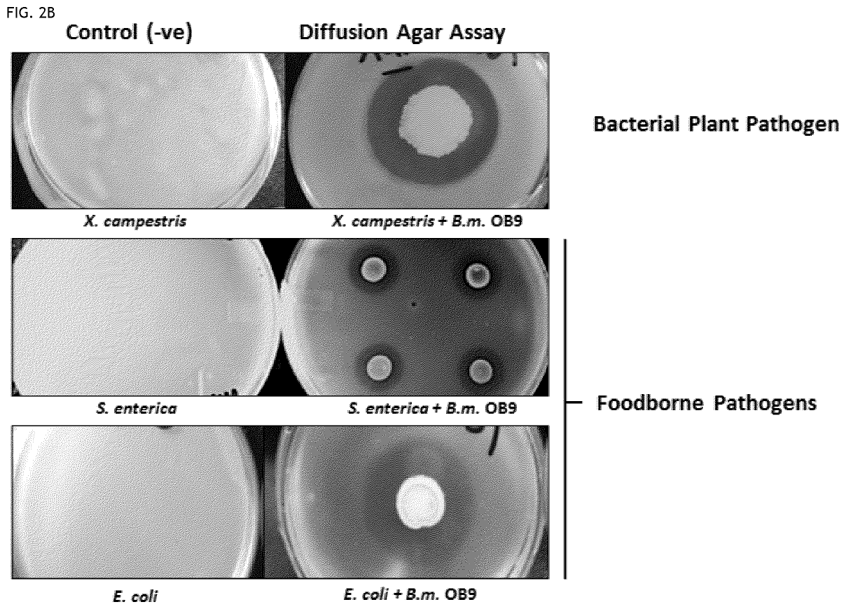

B. methylotrophicus OB9 was effective against all tested bacteria and fungi with varying degrees of antimicrobial activity (Table V, below; FIG. 2). More specifically, live cultures of B. methylotrophicus OB9 inhibited the growth of 23 Salmonella serovars (environmental, clinical and foodborne isolates), 4 strains of E. coli, 10 environmentally isolated Enterobacteriaceae strains and an X. campestris pv. Vitians isolate (Table V, below, FIGS. 2B-D) in agar diffusion assays, as visualized by a clear zone around the B. methylotrophicus OB9 colony that developed over 24-48 h. The diameter of the inhibition zone ranged from 3-6 mm and with some E. coli and Salmonella serovars a clear zone with double rings was observed (Table V, below), indicating the potency of one or more antimicrobials. The greatest zones of inhibition (6 mm in diameter) in response to B. methylotrophicus OB9 were observed in trials with X. campestris pv. Vitians and Salmonella serovar I:Rough-O:e,h:e,n (FIGS. 2B and C). Other Salmonella serovars I:4,5,12:b:-,I:Rough-O:-:e,n,x, Hartford, and Heidelberg) were also highly sensitive to B. methylotrophicus OB9, exhibiting clearing zones of 5 mm.

Interestingly, the biofilm-forming phenotypes (i.e., rdar—denoting the presence of the extracellular polysaccharide, cellulose, and curli fimbriae; saw—denoting the absence of cellulose and curli; Table III, above) of the Salmonella strains did not affect the ability of B. methylotrophicus OB9 to elicit zones of clearing. Even all of the control S. enterica Typhimurium strains that express both cellulose and curli (UMR1), only cellulose (MAE14), only curli fimbriae (MAE299) or neither component (MAE775) had a measurable zone of clearing around the B. methylotrophicus OB9 colony on the plates. These results suggest that the antimicrobial activities of B. methylotrophicus OB9 are not significantly affected by the biofilm barrier that bacterial cells use to exclude other antimicrobial agents (including disinfectants and antibiotics).

The widespread effectiveness of B. methylotrophicus OB9 is further exemplified by its ability to also inhibit the growth of plant pathogenic fungi Rhizoctonia solani and Botrytis cinerea, as observed in the dual confrontation assays (Table V, below; FIG. 2A).

TABLE V
Antagonistic activity of OB9 cultures against bacteria and fungi

| | | | OB9 |
|---|---|---|---|
| | | | Live |
| S. NO | Strain ID | Identity | cells |
| 1 | UMR1 | Salmonella typhimurium | +++ |
| 2 | MAE14 | S. typhimurium | + |
| 3 | MAE299 | S. typhimurium | + |
| 4 | MAE775 | S. typhimurium | ++ |
| 15 | SC01 | Serotype 1: 4, 5, 12: b: | +++* |
| 16 | SC16 | Serotype I: Rough-O: : e, n, x | +++* |
| 17 | SC04 | Serotype Braenderup | ++ |
| 18 | SCS7 | Serotype Typhimurium | + |
| 19 | SC110 | Serotype I: Rough-O: y: e, n, x | ++ |
| 20 | SC12 | Serotype I: Rough-O: e, h | + |
| 21 | SC18 | Serotype Hartford | +++* |
| 22 | SCS2 | Serotype: Rough-O: e, h: e, | ++++ |
| 23 | WTCR5 | Serotype 1: 6, 7: r | + |
| 24 | WTCR6 | Serotype Stanley | + |
| 25 | WTCR9 | Serotype InfaNtis | ++ |
| 26 | WTCR30 | Serotype- Schwarzengrund | + |
| 27 | WTCR22 | Serotype Thompson | ++ |
| 28 | WTC27 | serotype Heidleberg | +++* |
| 29 | WTC28 | Serotype Monschaui | + |
| 30 | WTCT4 | Serotype Heidleberg | ++ |
| 31 | S. agona | PARC#5 S. agona | ++ |
| 32 | SL1 | S. Newport | ++ |
| 33 | SL2 | S. Hartford | ++ |
| 34 | B07.007 | Xanthomonas hortorum | ++++ |
| 35 | E3-6 | Escherichia coli | + |
| 36 | E10-6 | E. coli | ++ |
| 37 | E14-6 | E. coli | +++* |
| 38 | E15-6 | E. coli | + |
| 39 | AG3-114 | Rhizoctonia solani | +++ |
| 40 | AG1-1(ROS-2A4) | R. solani | + |
| 41 | F014 | Botrytis cinerea | + |

Clearing zone of and above 3 mm is considered as positive; + clearing zone of 3 mm, ++ clearing zone of 4 mm, +++ 5 mm, ++++ 6 mm.
** Double ring of clearing zone.

### EXAMPLE 5: FRACTIONATION OF BIOSURFACTANT FROM B. METHYLOTROPHICUS OB9

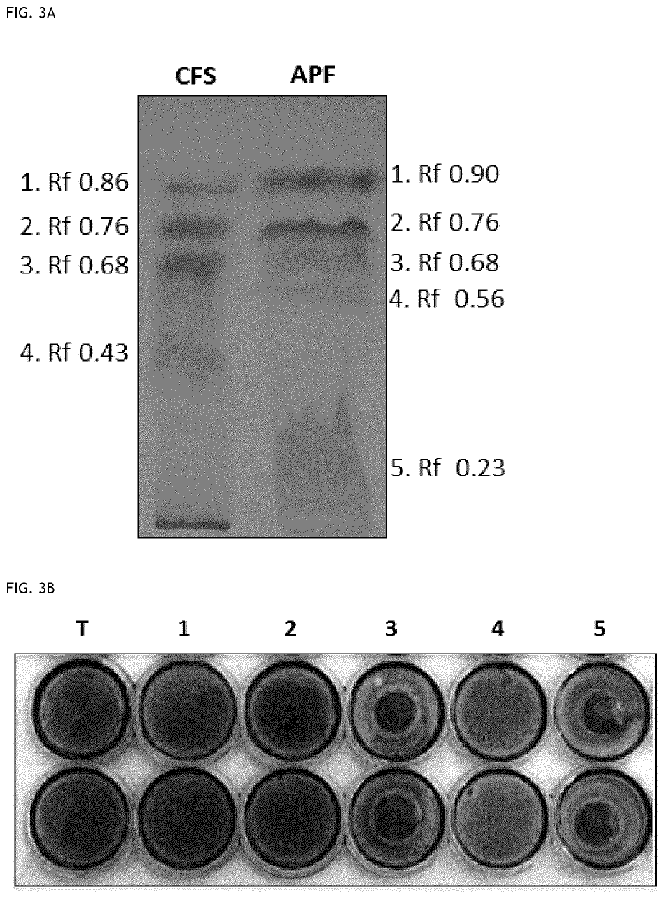

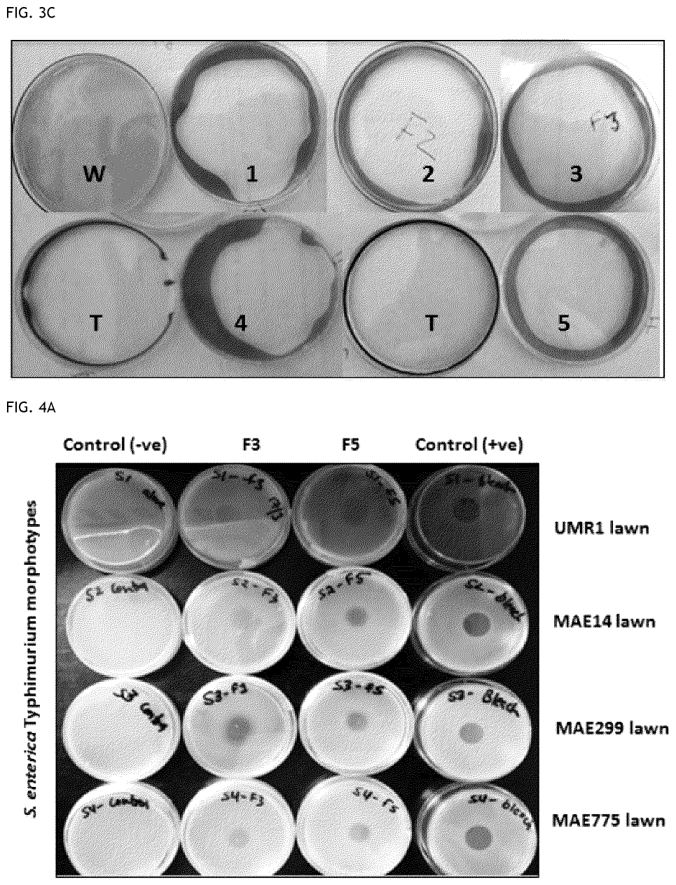

Biosurfactants were partially purified from culture filtrates of B. methylotrophicus OB9 grown in BHM broth enriched with oil. The purification procedure consisted of HCl precipitation, concentration of the precipitate by freeze-drying, dissolution in methanol-chloroform and analysis by TLC (FIG. 3A). Fractionation of cell free supernatant (CFS) yielded 4 fractions (1-4), while the acid precipitate fraction (APF) separated into 5 parts. Between the CFS and APF samples, fractions 2 and 3 from the respective samples had identical Rf values (0.68 and 0.23) (FIG. 3A).

### EXAMPLE 6: BIOSURFACTANT AND ANTIMICROBIAL ACTIVITY OF THE ACID PRECIPITATE FRACTION (APF) OF B. METHYLOTROPHICUS STRAIN OB9

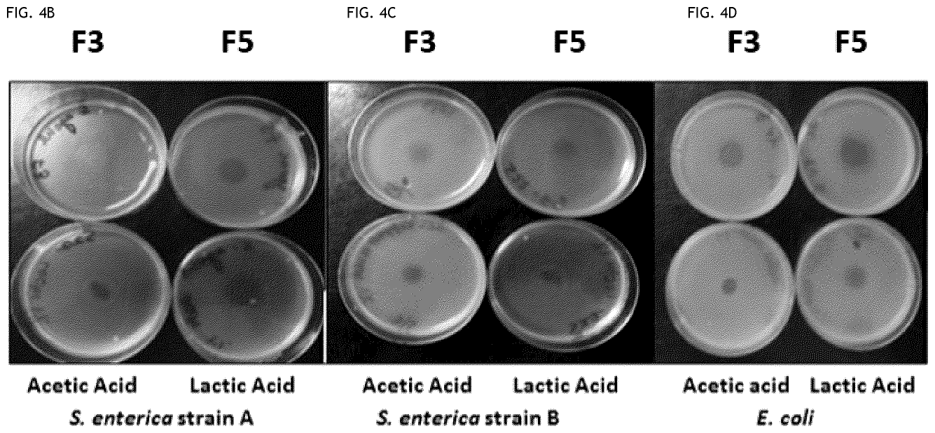

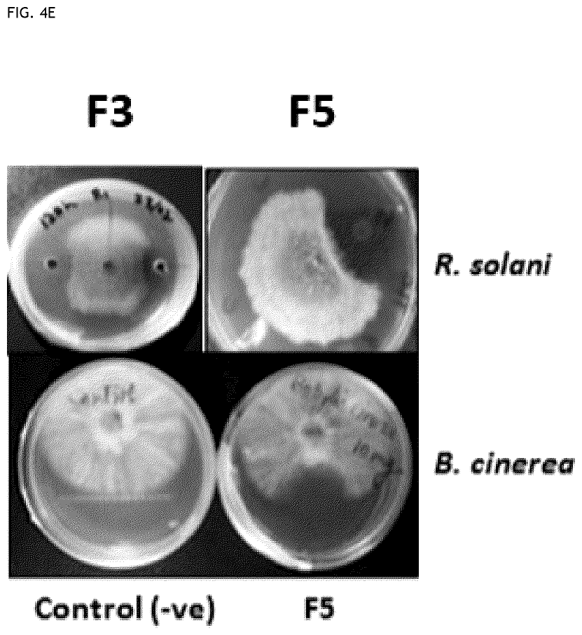

Only APF fractions (1-5) were assayed for biosurfactant and antimicrobial activities due to their improved purity and concentration relative to the CFS samples. All of the 5 TLC purified fractions of APF showed biosurfactant activity in the drop-collapse and oil-spreading assays (FIGS. 3B-3C). Fractions 3 and 5 had strong antimicrobial activities in diffusion agar plates against 13 test bacteria. This test panel included six Salmonella enterica serovars originating from the environment (SCS2), human fecal matter (SL1), or contaminated fresh produce (S. agoni Parc#5), as well as strains of E. coli and X. campestris (Data not shown) (a plant pathogenic bacterium) (Table VI, below; FIGS. 4A-D). Additionally, the APF fractions were moderately effective against two fungal plant pathogens (R. solani and B. cinerea) (Table VI, below; FIG. 4E); thereby indicating that these fractions have widespread microbial bioactivity. Furthermore, the size of the inhibition zones produced by the APF fractions (especially fraction 5) was comparable to that produced by the positive controls (Control (+ve), bleach (FIG. 4A) or common organic acids (lactic and acetic acid controls; FIGS. 4B-D) used as disinfectants and antimicrobials. Interestingly, the crude APF fraction (i.e., prior to TLC separation into fractions 3 (F3) and 5 (F5)) exhibited weak antimicrobial activity against the 13 bacteria and only one of the fungi (Table VI, below). Thus, isolation and partial enrichment of these fractions has increased their relative concentrations and/or efficacy.

TABLE VI
Microbial activity of the B. methylotrophicus OB9 fractions, organic
acids and bleach against various pathogenic bacteria and fungal strains

| | | | | | TLC Fractions | | Organic Acids^{a} | | | | Bleach |
|---|---|---|---|---|---|---|---|---|---|---|---|
| NO | Strain ID | Identity | Live cells | APF | F3 | F5 | AA | LA | FA | CA | 1.3% |
| 1 | UMR1 | Salmonella enterica Typhimurium | +++ | + | ++ | +++ | ++ | + | +++ | + | ++ |
| 2 | MAE14 | S. enterica Typhimurium | + | + | ++ | ++ | ++ | ++ | +++ | + | ++ |
| 3 | MAE299 | S. enterica Typhimurium | + | + | +++ | ++ | ++ | + | +++ | + | ++ |
| 4 | MAE775 | S. enterica Typhimurium | ++ | + | ++ | ++ | +++ | + | +++ | + | ++ |
| 5 | SCS2 | S. enterica I: Rough-O: e, h: e, n | ++++ | + | ++ | ++ | ++ | + | +++ | + | ++ |
| 6 | PARC#5 | S. enterica Agona | ++ | + | + | ++ | ++ | + | +++ | + | ++ |
| 7 | SL1 | S. enterica Newport | ++ | + | ++ | ++ | ++ | + | +++ | + | ++ |
| 8 | SL2 | S. enterica Hartford | ++ | + | ++ | ++ | ++ | + | +++ | + | ++ |
| 9 | B07.007 | Xanthomonas campestris | ++++ | + | ++ | +++ | +++ | ++ | +++ | ++ | ++ |
| 10 | E3-6 | Escherichia coli | + | + | ++ | ++ | ++ | + | +++ | + | ++ |
| 11 | E10-6 | E. coli | ++ | + | ++ | ++ | ++ | + | +++ | + | ++ |
| 12 | E14-6 | E. coli | +++^{b} | + | ++ | ++ | ++ | + | +++ | + | ++ |
| 13 | E15-6 | E. coli | + | + | ++ | ++ | ++ | + | +++ | + | ++ |
| 14 | AG3 (114) | Rhizoctonia solani | +++ | + | + | ++ | ++ | + | +++ | + | ++ |
| 15 | AG1-1A (ROS-2A) | R. solani | + | − | + | + | ++ | + | +++ | + | ++ |
| 16 | F014 | Botrytis cinerea | + | − | + | + | ++ | + | +++ | + | ++ |

^{a}Various organic acids (1%) tested as controls: AA, Acetic acid; LA, lactic acid; FA, formic acid; CA, citric acid.
^{b}Double ring of clearing zone. (+) clearing zone of 3 mm; (++) clearing zone of 4 mm; clearing zone of 5 mm (+++); clearing zone of 6 mm. (++++)

### EXAMPLE 7: SUPPRESSION OF SALMONELLA AND XANTHOMONAS GROWTH DURING CO-CULTURE WITH B. METHYLOTROPHICUS OB9

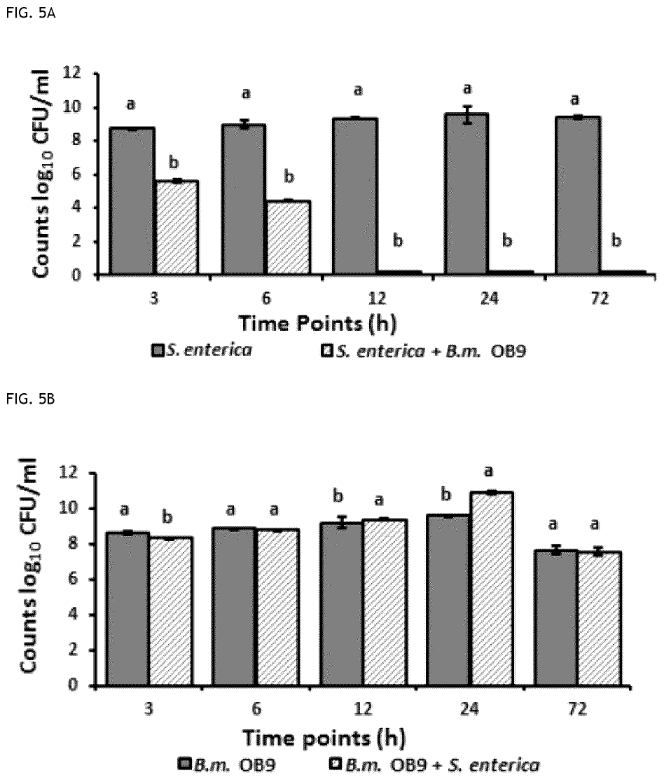

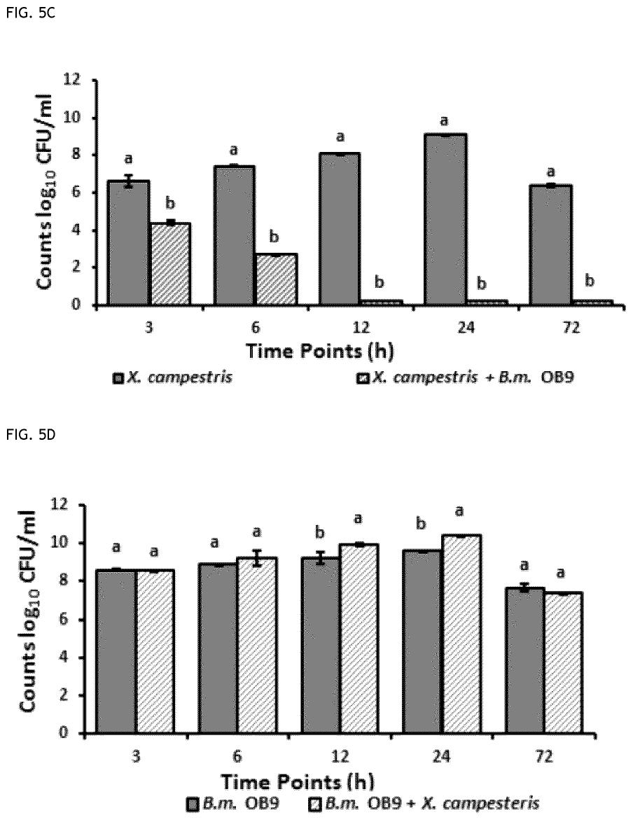

Salmonella cell numbers in liquid co-cultures with B. methylotrophicus OB9 significantly (P<0.05) decreased at 3 h (31-fold) and 6 h (45-fold) after incubation compared to growth controls (FIG. 5A). Similarly, X. campestris was 5 recovered 3 h and 6 h after incubation in co-cultures, with significant decreases of 22- and 47-fold in cell number, respectively (FIG. 5C). Furthermore, neither S. enterica Newport nor X. campestris were retrieved from co-cultures after 12 h or more of incubation (FIGS. 5A and C), while B. methylotrophicus OB9 continued to grow at a comparable rate to cultures of B. methylotrophicus OB9 alone (FIGS. 5B and D). To rule out the possibility of nutrient competition as a source of the antagonistic behaviour, media was replaced every 10 h, but this did not affect results.

### EXAMPLE 8: RECOLONIZATION, INTERNALIZATION, AND DETECTION OF BACTERIAL ISOLATES IN PLANT TISSUES

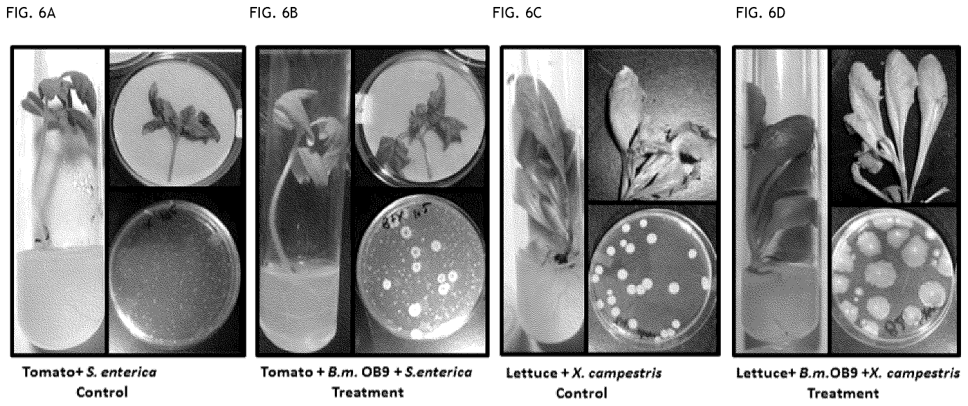

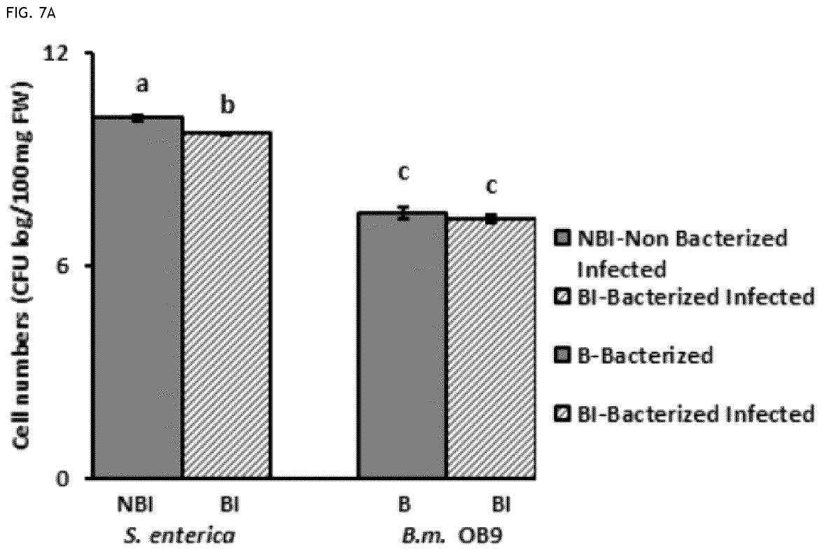

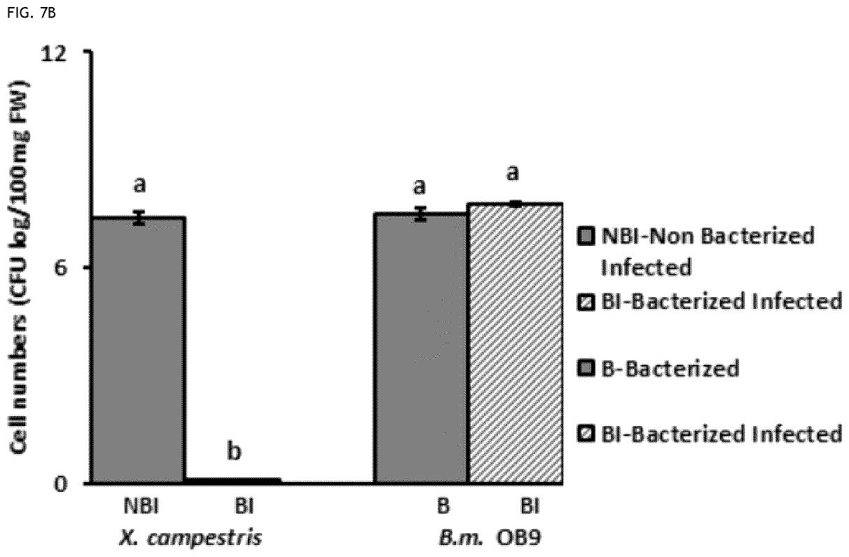

The surface-sterilization method combined with the imprint technique ensured that the endophytic colonization numbers reflected only the cells within the interior of the plant tissues (i.e., bacterized), as non-colonized plants did not yield culturable bacterial colonies (data not shown). Subsequent isolation and quantification of B. methylotrophicus (OB9) following surface sterilization demonstrated that the strain can travel from the roots to the stems and leaves and develop sustained endophytic populations in plant tissues of tomato (log₁₀ 7) and lettuce (log₁₀ 8) grown under gnotobiotic conditions even after the plants were challenged with Salmonella and Xanthomonas strains (FIGS. 6A-D and 7A-B).

### EXAMPLE 9: IMPACT OF PLANTS BACTERIZED WITH B. METHYLOTROPHICUS OB9 ON THE GROWTH OF SALMONELLA AND XANTHOMONAS

Tomato and lettuce leaves of B. methylotrophicus OB9 bacterized plants were detached and placed separately into petri plates lined with sterile filter paper that was moistened with sterile distilled water. An aliquot (100 μL) of each of Salmonella enterica Newport SL1 and X. campestris B07.007 inoculum (10⁶ cells mL⁻¹) was spread with a disposable spreader (Fischer Scientific Ltd) onto the surface of a tomato or lettuce leaf (S. enterica Newport SL1 and X. campestris B07.007, respectively). Non-bacterized leaves of tomato and lettuce were inoculated in the same manner and served as negative controls. All plates were sealed with parafilm and incubated at 22° C. Recovery of bacterial strains was determined by cell count after 72 h. Leaf tissue (100 mg) from infected and bacterized or non-bacterized plants was macerated in distilled water and serially diluted (10⁻¹ to 10⁻⁷) and aliquots from each dilution were plated on LBA plates and bacterial cell counts were estimated and reported as log₁₀ mL⁻¹. Detection of Salmonella on tomato leaves was confirmed by PCR with Salmonella specific primers ST11 (5′-GC CAA CCA TTG CTA AAT TGG CGC A-3′) (SEQ ID NO: 116) and ST15 (5′-GGT AGA AAT TCC CAG CGG GTA CTG-3′) (SEQ ID NO: 117) using an annealing temperature of 57° C. with PCR conditions described previously (Soumet et al., 1999). Detection of X. Campestris on lettuce leaves was confirmed using primers XcpM1 (5′-ACGCGCTACCAAAAGGCAAAGAG-3′) (SEQ ID NO: 118) and XcpM2 (5′-GATCTGCGGTTGTCCTGAAGATTGG-3′)(SEQ ID NO: 119) in conventional PCR assays (Sulzinski et al., 1996).

Leaves of B. methylotrophicus (OB9) bacterized tomato and lettuce plants challenged with Salmonella and Xanthomonas appeared healthy compared to challenged non-bacterized plants (FIGS. 6A-D). There was a significant (P<0.05) reduction of Salmonella cells (44% or 4.4-fold decrease) recovered from bacterized tomato leaves after 72 h (FIGS. 6A-B and 7A) compared to challenged non-bacterized leaves. In the case of Xanthomonas, there was no growth as reflected by complete absence of cells from bacterized lettuce tissues after 72 h (FIGS. 6B-C and 7B). Amplification of genomic DNA extracted from bacterized leaf samples using designed B. methylotrophicus OB9 specific primers gave the expected band size of 565 bp in leaf and root tissue samples (data not shown). Similarly, amplification of Salmonella from bacterized plants gave the expected band size of 429 bp, so the presence/absence of these bacteria was confirmed by this secondary method (data not shown). PCR amplification of Xanthomonas DNA was not successful using the XcpM 1/XcpM2 primers so this organism could not be detected in coated and challenged lettuce leaves by this secondary method.

### EXAMPLE 10: DIRECT INTERACTION OF B. METHYLOTROPHICUS OB9 WITH SALMONELLA ENTERICA NEWPORT SL1 OR WITH X. CAMPESTRIS B07.007 ON LEAF SURFACES

Tomato and lettuce leaves were submerged in 100 mL (10⁶ cells mL⁻¹) of B. methylotrophicus OB9 in a petri plate for 30 s and then left for 2 h. Aliquots (100 μL) of each of Salmonella and Xanthomonas strains (10⁶ cells mL⁻¹) were spread on the leaves, and incubated in petri plates at 22° C. Inoculums of Salmonella or Xanthomonas strains were also spread on non-inoculated tomato or lettuce leaves and served as experimental controls. After 72 h of incubation, recovery of bacterial cells from the leaves was determined by tissue maceration and cell count determination as described above.

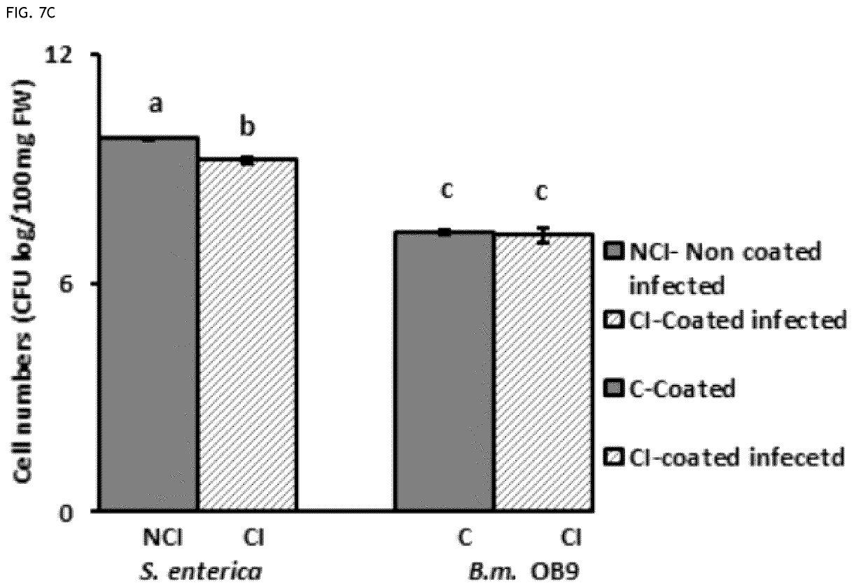

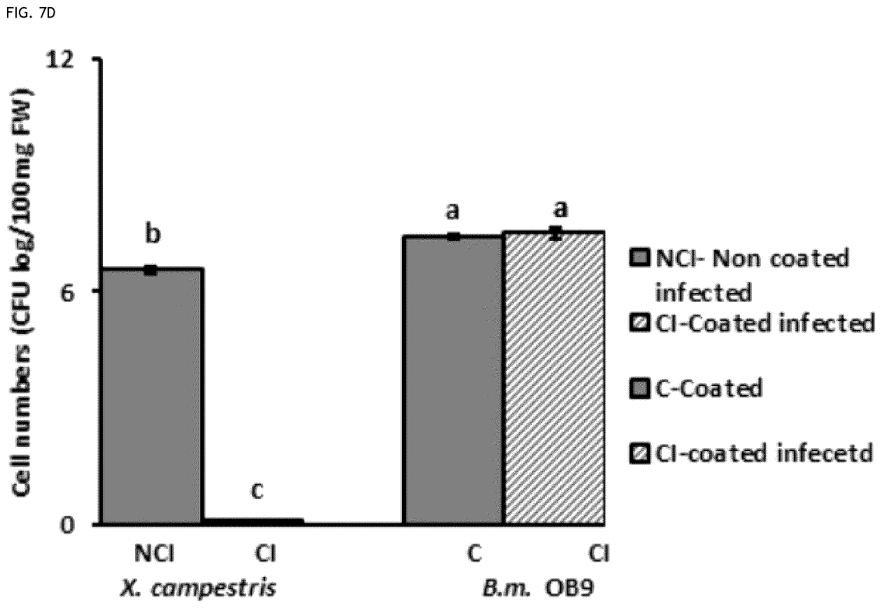

In this experiment, where tomato and lettuce leaves were coated with B. methylotrophicus OB9 (as opposed to bacterized as in Example 9) and challenged with Salmonella and Xanthomonas, similar trends were noted to the bacterized experiment. Recovery of Salmonella cell-numbers from these leaves was significantly decreased by 54% (5.4-fold decrease) compared to leaves treated with Salmonella alone (FIG. 7C). As in the bacterized experiment, Xanthomonas was not recovered in the B. methylotrophicus OB9 coating experiments either (FIG. 7D).

The scope of the claims should not be limited by the embodiments set forth in the examples but should be given the broadest interpretation consistent with the description as a whole.

### REFERENCES

Akbari, S., Abdurahman, N. H., Yunus, R. M., Fayaz, F., and Alara, O. R. (2018). Biosurfactants—a new frontier for social and environmental safety: a mini review. Biotechnology Research Innovation. 2, 81-90.
Alajlani, M., Shiekh, A., Hasnain, S., and Brantner, A. (2016). Purification of bioactive lipopeptides produced by Bacillus subtilis strain BIA. Chromatographia 79, 1527-1532.
Barak, J., Whitehand, L., and Charkowski, A. (2002). Differences in attachment of Salmonella enterica serovars and Escherichia coli 0157: H7 to alfalfa sprouts. Appl. Environ. Microbiol. 68, 4758-4763.
Brooijmans, R. J., Pastink, M. I., and Siezen, R. J. (2009). Hydrocarbon-degrading bacteria: the oil-spill clean-up crew. Microb. Biotechnol. 2, 587-594.
Burkholder, P. R., Evans, A. W., Mcveigh, I., and Thornton, H. K. (1944). Antibiotic activity of lichens. Proc. Natl. Acad. Sci. U. S. A. 30, 250.
Cai, M., Nie, Y., Chi, C. Q., Tang, Y. Q., Li, Y., Wang, X. B., Liu, Z. S., Yang, Y., Zhou, J., and Wu, X. L. (2015). Crude oil as a microbial seed bank with unexpected functional potentials. Sci. Rep. 5, 16057.
Cawoy, H., Debois, D., Franzil, L., De Pauw, E., Thonart, P., and Ongena, M. (2015). Lipopeptides as main ingredients for inhibition of fungal phytopathogens by Bacillus subtilis/amyloliquefaciens. Microb. Biotechnol. 8, 281-295.
Chandankere, R., Yao, J., Choi, M. M., Masakorala, K., and Chan, Y. (2013). An efficient biosurfactant-producing and crude-oil emulsifying bacterium Bacillus methylotrophicus USTBa isolated from petroleum reservoir. Biochem. Eng. J. 74, 46-53.
Chaprão, M. J., Rita De Cássia, F., Rufino, R. D., Luna, J. M., Santos, V. A., and Sarubbo, L. A. (2018). Production of a biosurfactant from Bacillus methylotrophicus UCP1616 for use in the bioremediation of oil-contaminated environments. Ecotoxicology. 27, 1310-1322.
D'aes, J., De Maeyer, K., Pauwelyn, E., and Höfte, M. (2010). Biosurfactants in plant-Pseudomonas interactions and their importance to biocontrol. Environ Microbiol Rep. 2, 359-372.
Debode, J., Maeyer, K. D., Perneel, M., Pannecoucque, J., Backer, G. D., and Höfte, M. (2007). Biosurfactants are involved in the biological control of Verticillium microsclerotia by Pseudomonasspp. J. Appl. Microbiol. 103, 1184-1196.
Deering, A. J., Mauer, L. J., and Pruitt, R. E. (2012). Internalization of E. coli O157: H7 and Salmonella spp. in plants: a review. Food. Res. Int. 45, 567-575.
Díaz De Rienzo, M. A., Stevenson, P., Marchant, R., and Banat, I. M. (2015). Antibacterial properties of biosurfactants against selected Gram-positive and-negative bacteria. FEMS Microbiol. Lett. 363, fnv224.
Fletcher, J., Leach, J. E., Eversole, K., and Tauxe, R. V. (2013). Human pathogens on plants: designing a multidisciplinary strategy for research. Phytopathology 103, 306-315.
Gagne-Bourgue, F., Aliferis, K., Seguin, P., Rani, M., Samson, R., and Jabaji, S. (2013). Isolation and characterization of indigenous endophytic bacteria associated with leaves of switchgrass (Panicum virgatum L.) cultivars. J. Appl. Microbiol. 114, 836-853.
Gagné-Bourque, F., Mayer, B. F., Charron, J.-B., Vali, H., Bertrand, A., and Jabaji, S. (2015). Accelerated growth rate and increased drought stress resilience of the model grass Brachypodium distachyon colonized by Bacillus subtilis B26. PLoS One. 10, e0130456.
Gibson, D., White, A., Snyder, S., Martin, S., Heiss, C., Azadi, P., Surette, M., and Kay, W. (2006). Salmonella produces an O-antigen capsule regulated by AgfD and important for environmental persistence. J. Bacteriol. 188, 7722-7730.
Heydari, A., and Pessarakli, M. J. J. O. B. S. (2010). A review on biological control of fungal plant pathogens using microbial antagonists. J. Biol. Sci. 10, 273-290.
Hsu, C.-K., and Micallef, S. A. (2017). Plant-mediated restriction of Salmonella enterica on tomato and spinach leaves colonized with Pseudomonas plant growth-promoting rhizobacteria. Int. J. Food Microbiol. 259, 1-6.
Huang, B., Lv, C., Zhuang, P., Zhang, H., and Fan, L. (2011). Endophytic colonisation of Bacillus subtilis in the roots of Robinia pseudoacacia L. Plant Biol. 13, 925-931.
Ibrahim, M., Ijah, U., Manga, S., Bilbis, L., and Umar, S. (2013). Production and partial characterization of biosurfactant produced by crude oil degrading bacteria. Int Biodeterior Biodegradation. 81, 28-34.
Jain, D., Collins-Thompson, D., Lee, H., and Trevors, J. (1991). A drop-collapsing test for screening surfactant-producing microorganisms. J. Microbiol. Methods 13, 271-279.
Jemil, N., Ayed, H. B., Hmidet, N., and Nasri, M. (2016). Characterization and properties of biosurfactants produced by a newly isolated strain Bacillus methylotrophicus DCS1 and their applications in enhancing solubility of hydrocarbon. World J. Microbiol. Biotechnol. 32, 175.
Jeukens, J., Kukavica-lbrulj, I., Freschi, L., Jabaji, S., and Levesque, R. C. (2015). Draft genome sequences of two lipopeptide-producing strains of Bacillus methylotrophicus. Genome Announc 3, e01176-01115.
Korenblum, E., Souza, D. B., Penna, M., and Seldin, L. (2012). Molecular analysis of the bacterial communities in crude oil samples from two Brazilian offshore petroleum platforms. Int. J. Microbiol. 2012.
Lenchi, N., Inceoğlu, Ö., Kebbouche-Gana, S., Gana, M. L., Lliros, M., Servais, P., and Garcia-Armisen, T. (2013). Diversity of microbial communities in production and injection waters of Algerian oilfields revealed by 16S rRNA gene amplicon 454 pyrosequencing. PLoS One. 8, e66588.
Lynch, M. F., Tauxe, R. V., Hedberg, C. W. (2009). The growing burden of foodborne outbreaks due to contaminated fresh produce: risks and opportunities. Epidemiol. Infect. 137, 307-315.
Magot, M., Ollivier, B., and Patel, B. K. (2000). Microbiology of petroleum reservoirs. Antonie Van Leeuwenhoek 77, 103-116.
Mani, P., Dineshkumar, G., Jayaseelan, T., Deepalakshmi, K., Kumar, C. G., and Balan, S. S. (2016). Antimicrobial activities of a promising glycolipid biosurfactant from a novel marine Staphylococcus saprophyticus SBPS 15. 3 Biotech. 6, 163.
Marchant, R., and Banat, I. M. (2012). Biosurfactants: a sustainable replacement for chemical surfactants? Biotechnol. Lett. 34, 1597-1605.
Marchut-Mikolajczyk, O., Drożdżyński, P., Pietrzyk, D., & Antczak, T. J. M. c. f. (2018). Biosurfactant production and hydrocarbon degradation activity of endophytic bacteria isolated from Chelidonium majus L. 17(1), 171.
McNees, C. R., Greenhut, I. V., Law, A. D., Saleem, M. and Moe, L. A. (2019). Life within the leaf: ecology and applications of foliar bacterial endophytes. In Endophytes for a Growing World, ed. T. R. Hodkinson, F. M. Doohan, M. J. Saunders and B. R. Murphy. (Cambridge: Cambridge University Press), Chapter 10. 208-230.
McSpadden Gardener, B. B. (2004). Ecology of Bacillus and Paenibacillus spp. in agricultural systems. Phytopathology. 94, 1252-1258.
Meena, K. R., and Kanwar, S. S. J. B. R. I. (2015). Lipopeptides as the antifungal and antibacterial agents: applications in food safety and therapeutics. Biomed Res. Int. 2015.
Milanov, D. S., Prunić, B. Z., Velhner, M. J., Pajić, M. L., and C̆abarkapa, I. S. (2015). RDAR morphotype: A resting stage of some Enterobacteriaceae. Food and Feed Research. 42, 43-50.
Moore, T., Globa, L., Barbaree, J., Vodyanoy, V., and Sorokulova, I. (2013). Antagonistic activity of Bacillus bacteria against food-borne pathogens. Probiotics Health 1, 1-6.
Mulligan, C. N. (2005). Environmental applications for biosurfactants. Environ. Pollut. 133, 183-198.
Murray, K., Wu, F., Shi, J., Jun Xue, S., Warriner, K., and Safety (2017). Challenges in the microbiological food safety of fresh produce: Limitations of post-harvest washing and the need for alternative interventions. Food Quality and safety. 1, 289-301.
Muthezhilan, R., Ragul, R., Pushpam, A. C., and Hussain, A. J. (2014). Production and Purification of Biosurfactant from Marine Yeast Isolated from Kelambakkam Salterns. Biosci., Biotechnol. Res. Asia. 11, 59-67.
Nicolas, O., Charles, M. T., Jenni, S., Toussaint, V., and Beaulieu, C. (2018). Relationships between Xanthomonas campestris pv. vitians population sizes, stomatal density and lettuce resistance to bacterial leaf spot. Can. J. Plant Pathol. 40, 399-407.
Nitschke, M., and Costa, S. (2007). Biosurfactants in food industry. Trends Food. Sci. Technol. 18, 252-259.
Obi, L. U., Atagana, H. I., and Adeleke, R. A. (2016). Isolation and characterisation of crude oil sludge degrading bacteria. SpringerPlus 5, 1946.
Parthipan, P., Preetham, E., Machuca, L. L., Rahman, P. K., Murugan, K., and Rajasekar, A. (2017). Biosurfactant and degradative enzymes mediated crude oil degradation by bacterium Bacillus subtilis A1. Front. Microbiol. 8, 193.
Perfumo, A., Rancich, I., and Banat, I. M. (2010). “Possibilities and challenges for biosurfactants use in petroleum industry,” in Biosurfactants. Springer), 135-145.
Phan, C.-W., Abu Bakar, N. F., and Hamzah, A. (2013). A comparative study on biosurfactant activity of crude oil-degrading bacteria and its correlation to total petroleum hydrocarbon degradation. Bioremediat. J. 17, 240-251.
Pillay, V., and Nowak, J. (1997). Inoculum density, temperature, and genotype effects on in vitro growth promotion and epiphytic and endophytic colonization of tomato (Lycopersicon esculentum L.) seedlings inoculated with a Pseudomonad bacterium. Can. J. Microbiol. 43, 354-361.
Porebski, S., Bailey, L. G., and Baum, B. R. (1997). Modification of a CTAB DNA extraction protocol for plants containing high polysaccharide and polyphenol components. Plant Mol. Biol. Rep. 15, 8-15.
Römling, U., Bian, Z., Hammar, M., Sierralta, W. D., and Normark, S. (1998). Curli fibers are highly conserved between Salmonella typhimurium and Escherichia coli with respect to operon structure and regulation. J. Bacteriol. 180, 722-731.
Roy, A. (2017). Review on the Biosurfactants: Properties, Types and its Applications. J. Fundam. Renew. Energy Appl. 8, 2.
Sachdev, D. P., and Cameotra, S. S. (2013). Biosurfactants in agriculture. Appl. Microbiol. Biotechnol. 97, 1005-1016.
Saleem, M., Meckes, N., Pervaiz, Z. H., & Traw, M. B. J. F. i. m. (2017). Microbial interactions in the phyllosphere increase plant performance under herbivore biotic stress. 8, 41.
Santos, D., Rufino, R., Luna, J., Santos, V., & Sarubbo, L. J. I. j. o. m. s. (2016). Biosurfactants: multifunctional biomolecules of the 21st century. 17(3), 401.
Santoyo, G., Moreno-Hagelsieb, G., Del Carmen Orozco-Mosqueda, M., and Glick, B. R. (2016). Plant growth-promoting bacterial endophytes. Microbiol. Res. 183, 92-99.
Schikora, A., Garcia, A. V., and Hirt, H. (2012). Plants as alternative hosts for Salmonella. Trends Plant Sci. 17, 245-249.
Scott, M., Rani, M., Samsatly, J., Charron, J.-B., and Jabaji, S. (2018). Endophytes of industrial hemp (Cannabis sativa L.) cultivars: identification of culturable bacteria and fungi in leaves, petioles, and seeds. Can. J. Microbiol. 64, 664-680.
Snyder, T. R., Boktor, S. W., and M'lkanatha, N. M. (2019). Salmonellosis Outbreaks by Food Vehicle, Serotype, Season, and Geographical Location, United States, 1998 to 2015. J. Food Prot. 82(7), 1191-1199.
Soumet, C., Ermel, G., Rose, V., Rose, N., Drouin, P., Salvat, G., and Colin, P. (1999). Identification by a multiplex PCR-based assay of Salmonella typhimurium and Salmonella enteritidis strains from environmental swabs of poultry houses. Lett. Appl. Microbiol. 29, 1-6.
Stepanović, S., Ćirković, I., Ranin, L., and Svabić-Vlahović, M. (2004). Biofilm formation by Salmonella spp. and Listeria monocytogenes on plastic surface. Lett. Appl. Microbiol. 38, 428-432.
Sulzinski, M., Moorman, G., Schlagnhaufer, B., and Romaine, C. (1996). Characteristics of a PCR-based assay for in planta detection of Xanthomonas campestris pv. pelargonii. J. Phytopathol. 144, 393-398.
Tahzibi, A., Kamal, F., and Mazaheri Assadi, M. (2004). Improved production of rhamnolipids by a Pseudomonas aeruginosa mutant. Iran. Biomed. J. 8, 25-31.
Thavasi, R. (2011). Microbial biosurfactants: from an environmental application point of view. J. Bioremediat. Biodegrad. 2.
Trias, R., Bañieras, L., Montesinos, E., and Badosa, E. (2008). Lactic acid bacteria from fresh fruit and vegetables as biocontrol agents of phytopathogenic bacteria and fungi. Int. Microbiol. 11, 231.
Varjani, S. J., and Gnansounou, E. (2017). Microbial dynamics in petroleum oilfields and their relationship with physiological properties of petroleum oil reservoirs. Bioresour. Technol. 245, 1258-1265.
Varjani, S. J., and Upasani, V. N. (2017). Critical review on biosurfactant analysis, purification and characterization using rhamnolipid as a model biosurfactant. Bioresour. Technol. 232, 389-397.
Walter, V., Syldatk, C., and Hausmann, R. (2010). “Screening concepts for the isolation of biosurfactant producing microorganisms.,” in Biosurfactants. Bioscience and Springer Science+Business Media), 1-13.
Wang, T., Sun, H., Mao, H., Zhang, Y., Wang, C., Zhang, Z., Wang, B., and Sun, L. (2014). The immobilization of heavy metals in soil by bioaugmentation of a UV-mutant Bacillus subtilis 38 assisted by NovoGro biostimulation and changes of soil microbial community. J. Hazard. Mater. 278, 483-490.
Wu, J.-Y., Yeh, K.-L., Lu, W.-B., Lin, C.-L., and Chang, J.-S. (2008). Rhamnolipid production with indigenous Pseudomonas aeruginosa EM1 isolated from oil-contaminated site. Bioresour. Technol. 99, 1157-1164.
Wulff, E. G., Mguni, C. M., Mortensen, C. N., Keswani, C. L., and Hockenhull, J. (2002). Biological control of black rot (Xanthomonas campestris pv. campestris) of brassicas with an antagonistic strain of Bacillus subtilis in Zimbabwe. Eur. J. Plant Pathol. 108, 317-325.
Xiao, M., Sun, S.-S., Zhang, Z.-Z., Wang, J.-M., Qiu, L.-W., Sun, H.-Y., Song, Z.-Z., Zhang, B.-Y., Gao, D.-L., and Zhang, G.-Q. (2016). Analysis of bacterial diversity in two oil blocks from two low-permeability reservoirs with high salinities. Sci. Rep. 6, 19600.
Xu, X., Liu, W., Tian, S., Wang, W., Qi, Q., Jiang, P., Gao, X., Li, F., Li, H., and Yu, H. (2018). Petroleum hydrocarbon-degrading bacteria for the remediation of oil pollution under aerobic conditions: A perspective analysis. Front. Microbiol. 9, 2885.
Yoshida, N., Yagi, K., Sato, D., Watanabe, N., Kuroishi, T., Nishimoto K., et. al. (2005). Bacterial communities in petroleum oil in stockpiles. J. Biosci. Bioeng. 99, 143-149.
Youssef, N. H., Duncan, K. E., Nagle, D. P., Savage, K. N., Knapp, R. M., and Mcinerney, M. J. (2004). Comparison of methods to detect biosurfactant production by diverse microorganisms. J. Microbiol. Methods. 56, 339-347.
Zhao, H., Shao, D., Jiang, C., Shi, J., Li, Q., Huang, Q et. al (2017). Biological activity of lipopeptides from Bacillus. App. Microbiol. Biotechnol. 101(15), 5951-5960.
Zogaj, X., Nimtz, M., Rohde, M., Bokranz, W., and Römling, U. (2001). The multicellular morphotypes of Salmonella typhimurium and Escherichia coli produce cellulose as the second component of the extracellular matrix. Mol. Microbiol. 39, 1452-1463.

1. Method of using a bacterium that is a Bacillus, Streptomyces, Microbacterium, Micrococcus, Rhodococcus, Pseudomonas, Arthrobacter or Staphylococcus and/or a biosurfactant-containing extract isolated from said bacterium, as an antimicrobial agent against at least one foodborne bacterial or fungal pathogen and/or at least one plant bacterial or fungal pathogen.

2. The method of claim 1, wherein the bacterium is (i) a Bacillus, or (ii) a Bacillus velezensis; or a Bacillus amyloliquefaciens.

3. (canceled)

4. The method of claim 1, wherein (a) the bacterium produces, or the biosurfactant-containing extract contains, at least 3 of bacilysin, difficidin, macrolactin H, bacillaene, bacillomycin D, fengycin, surfactin and bacillibactin; and/or (b) the bacterium encodes at least 10 of SEQ ID NOs: 1-111.

5. (canceled)

6. The method of claim 1, wherein the bacterium is a Bacillus velezensis OB9.

7. The method of claim 1, wherein the bacterial or fungal pathogen is (a1) a Salmonella, an Escherichia coli, an Enterobacteriaceae, an Xanthomonas campestris, a Rhizoctonia solani or a Botrytis cinereal; or (a2) a Salmonella or a Xanthomonas campestris.

8. (canceled)

9. The method of claim 1, wherein the at least one bacterial or fungal pathogen comprises at least one foodborne pathogen and at least one plant pathogen.

10. The method of claim 1, wherein the at least one bacterial or fungal pathogen comprises a plant pathogen, and wherein the method comprises bacterizing a plant or coating food or a plant with the bacterium, preferably wherein the plant is a tomato plant or a lettuce plant.

11. (canceled)

12. The method of claim 1, wherein the at least one bacterial pathogen comprises a biofilm producing bacterial pathogen, preferably a Salmonella such as a Salmonella enterica.

13. (canceled)

14. (canceled)

15. The method of claim 1, wherein the method uses the biosurfactant-containing extract isolated from said bacterium, preferably an acid precipitate fraction of the biosurfactant-containing extract.

16. (canceled)

17. A plant or plant part bacterized or coated with a Bacillus or a biosurfactant-containing extract isolated from the Bacillus.

18. The plant or plant part of claim 17, wherein the Bacillus is a Bacillus velezensis.

19. The plant or plant part of claim 17, wherein (i) the Bacillus produces, or the biosurfactant-containing extract contains at least 3 of bacilysin, difficidin, macrolactin H, bacillaene, bacillomycin D, fengycin, surfactin and bacillibactin; or (ii) the Bacillus encodes at least 10 of SEQ ID NOs: 1-111.

20. (canceled)

21. The plant or plant part of claim 17, wherein the Bacillus is Bacillus velezensis OB9.

22. The plant or plant part of claim 17, which is a tomato or a lettuce plant, plant part or cell.

23. A method of protecting a plant or plant part against at least one bacterial or fungal pathogen comprising bacterizing or coating the plant or plant part with a Bacillus or a biosurfactant-containing extract isolated from the Bacillus, wherein the Bacillus produces or the extract contains at least 3 of bacilysin, difficidin, macrolactin H, bacillaene, bacillomycin D, fengycin, surfactin and bacillibactin.

24. The method of claim 23, wherein the Bacillus is a Bacillus velezensis.

25. The method of claim 23, wherein (A) (i) the Bacillus encodes at least 10 of SEQ ID NOs: 1-111; and/or (B) the method comprises (i) bacterizing or coating the plant; or (ii) bacterizing or coating the plant part.

26. The method of claim 23, wherein the Bacillus is a Bacillus methylotrophicus OB9.

27. (canceled) 28. (canceled)

29. The method of claim 23, wherein the at least one bacterial or fungal pathogen comprises a Salmonella, an Escherichia coli, an Enterobacteriaceae, an Xanthomonas campestris, a Rhizoctonia solani or a Botrytis cinereal.

30. The method of claim 23, wherein the at least one bacterial pathogen comprises a Salmonella or a Xanthomonas campestris.
